(19) United States
(12) Patent Application Publication
(10) Pub. No.: US 2006/0046972 A1
(43) Pub. Date: Mar. 2, 2006

(54) Cytotoxic nucleoside analog compound 003 for treating cancer

(75) Inventors: Fatih M. Uckun, White Bear Lake, MN (US); Taracad Venkatachalam, Maplewood, MN (US)

Correspondence Address: MERCHANT & GOULD PC, P.O. BOX 2903, MINNEAPOLIS, MN, 55402-0903, US

(73) Assignee: Parker Hughes Institute, Roseville, MN (US)

(21) Appl. No.: 11/216,640

(22) Filed: Aug. 31, 2005

Related U.S. Application Data
(60) Provisional application No. 60/606,158, filed on Aug. 31, 2004.
(60) Provisional application No. 60/606,933, filed on Sep. 2, 2004.

(51) Int. Cl. A61K 31/7072

(52) U.S. Cl. 514/51

(57) ABSTRACT

The present invention provides pharmaceutical compositions comprising Compound 003 or metabolites thereof in combination with one or more carboxylesterase inhibitors. The invention provides methods for inhibiting cellular proliferation associated with proliferative cell disorders in a subject by administering Compound 003 or metabolites thereof. The invention also provides methods for arresting the cell cycle. Methods of inhibiting proliferation of cells for treatment of cancer by administering Compound 003 are described.

### PRIORITY

This application claims priority to a U.S. Provisional App. No. 60/606,158, filed Aug. 31, 2004 and U.S. Provisional App. No. 60/606,933, filed Sep. 2, 2004, both hereby incorporated by reference in their entirety.

### BACKGROUND

Breast cancer is the most common cancer in women with more than 200,000 new cases and more than 40,000 deaths each year (National Cancer Institute, http:H/srab.cancer.gov/devcan). It is the second leading cause (after lung cancer) of cancer related deaths in women. Although the majority of patients with metastatic breast cancer will experience an initial response, survival is only modestly improved with contemporary chemotherapy programs. Front-line cytotoxic chemotherapy of metastatic breast cancer with the most effective regimens offers a median duration of response of only 8 months. Once patients progress after the front-line therapy, the response rate is only 20-35% for second-line combination chemotherapy. One of the major challenges in the treatment of breast cancer is to cure patients who have metastatic disease.

The Her2/neu oncogene has been mapped to the long arm of chromosome 17 and encodes a transmembrane glycoprotein with tyrosine kinase activity. Overexpression of HER2 is observed in 25-30% of breast cancer patients and is associated with absence of estrogen receptor expression, aneuploidy, poorly differentiated tumor grade, and higher proliferative rate. In breast cancer, expression of the HER-2/neu oncogene is a significant and independent indicator for recurrence and poor relapse-free survival. Development of new agents for high risk patients such as HER2 positive premenopausal breast cancer patients who are more likely to fail the existing treatment regimens or advanced metastatic breast cancer patients drugs has emerged as an exceptional focal point for translational research aimed at designing more effective treatment strategies for breast cancer.

Zidovudine/AZT and its derivatives have been found to exhibit anti-cancer activity especially in combination with inhibitors of thymidylate synthase such as 5-fluorouracil, the anti-tubulin agent paclitaxel (taxol), or interferon (IFN). See, for example, Barnes, et al. (2004) Endocr Relat Cancer. 11, 85; Gee, et al. (2001) Int J Cancer. 95, 247; and Lindley, C., (2002) J Am Pharm Assoc (Wash). 42, S30. In some studies, zidovudine has been shown to cause telomerase inhibition in breast cancer, cervical cancer and colon cancer cells. Zidovudine has also been used to enhance the chemosensitivity of cancer cells to cisplatin.

Although zidovudine has been suggested as a cancer therapeutic, its potential has not been clinically achieved. It would be very useful to identify new molecules for the treatment of cancer.

### SUMMARY OF THE INVENTION

The present invention provides pharmaceutical compositions comprising Compound 003 or metabolites thereof in combination with one or more carboxylesterase inhibitors. The invention also provides methods for inhibiting cellular proliferation associated with proliferative cell disorders in a subject by administering Compound 003 or metabolites thereof.

The invention provides methods for arresting the cell cycle, including inhibiting mitotic spindle formation, inhibiting mitosis of cells. In particular, the methods of the invention include inhibiting mitosis of breast cancer cells, killing breast cancer cells, and treating breast cancer by administering Compound 003. Methods of inhibiting proliferation of cells for treatment of cancer by administering Compound 003 are described.

### DESCRIPTION OF THE FIGURES

FIG. 1 shows the effect of Compound 003 on aster formation in isolated centrosomes. The organization and size of asters grown from centrosomes isolated from an EBV-transformed B-lymphoblastoid cell line were compared. The presence of Compound 003 inhibited the ability of the centrosomes to nucleate and sufficiently grow microtubule asters. Two representative asters from each experiment are shown. A&B: vehicle; C&D: Compound 003 100 μM.

FIG. 2 shows the effect of Compound 003 on the mitotic spindle of cancer cells. Confocal laser scanning microscopy experiments demonstrated that Compound 003 (100 μM) prevents the normal process of microtubule assembly and induced aberrant multipolar spindle formation in intact BT20 breast cancer cells (A&B), MOLT-3 T-lymphoma cells (C&D), DU145 prostate cancer cells (E&F) and U373 glioblastoma cells (G&H).

FIG. 3 shows the effect of Compound 003 on the mitotic spindle of prostate cancer cells. Confocal laser scanning microscopy images of PC-3 prostate cancer cells treated with vehicle (A&B) or 100 μM Compound 003 (C&D). Compound 003 prevents the normal process of microtubule assembly and induced aberrant multipolar spindle formation.

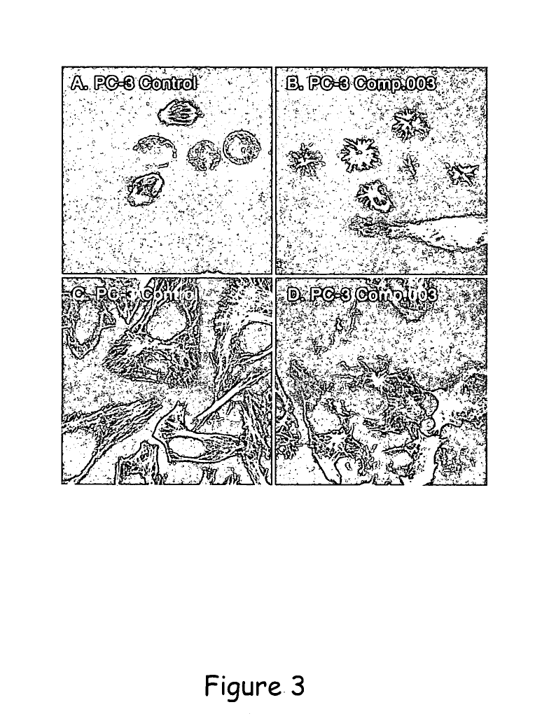

FIG. 4 shows G₂M cell cycle arrest in lymphoma cells administered Compound 003. Various intact propidium iodide-labeled lymphoma cells were analyzed by DNA flow cytometry to screen for possible cell cycle arrest. Compound 003 caused G2 arrest in DT40, chicken B lymphoma; MOLT-3, human T lymphoma; and DAUDI, human Burkitt's lymphoma cell lines as reflected by a marked increase of the proportion of nuclei in the G2M peak of the DNA histograms with a concomitant decrease of the proportion of nuclei in the G0/1 peak. Quantitative DNA analysis was performed on a Becton Dickenson Calibur flow cytometer equipped with a Consort 40 computer using the COTFIT program, which includes CELLCY, a cell cycle distribution function that fits DNA content histograms and calculates the percentages of cells in G, S, and GM phases of the cell cycle.

FIG. 5 shows G₂M cell cycle arrest in human leukemia cells administered Compound 003. Intact propidium iodide-labeled human infant leukemia (RS4; 11), human pre-B acute lymphoblastic leukemia (NALM-6) and human T leukemia/lymphoma (JURKAT) cell lines were analyzed by DNA flow cytometry as described in the Materials and Methods section.

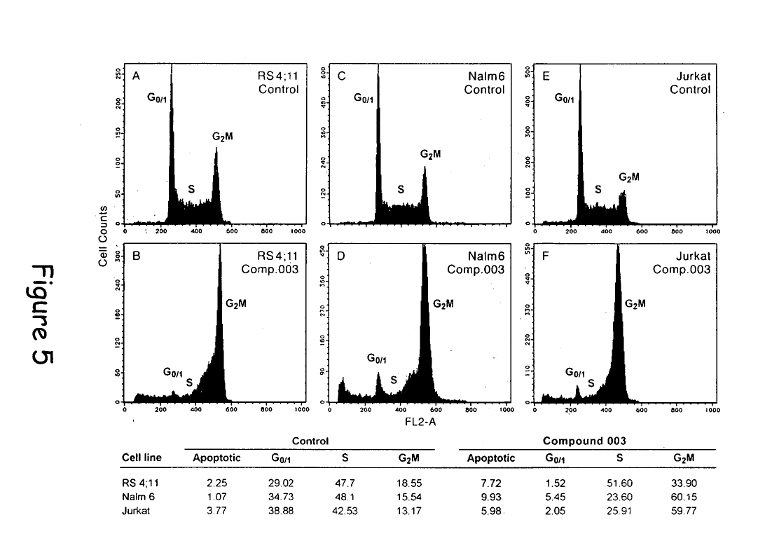

FIG. 6 shows a Protective Effect of Compound 003 in a murine model of Her 2 positive breast cancer. Virgin MMTV/Neu transgenic mice were treated with Compound 003 that was added to the daily food of the mice at an average daily dose level of 50 mg/kg/day for the duration of the 600-day experiment. Mice were screened for the tumor formation every other day. Tumor-free survival was analyzed using life table statistics as previously reported. The protective anti-cancer activity of Compound 003 was evaluated according to the rise of the survival rate and prolongation of the tumor-free survival of the experimental animals as compared with the control animals. Statistical Significance was determined using the Kaplan Meier Log-Rank test.

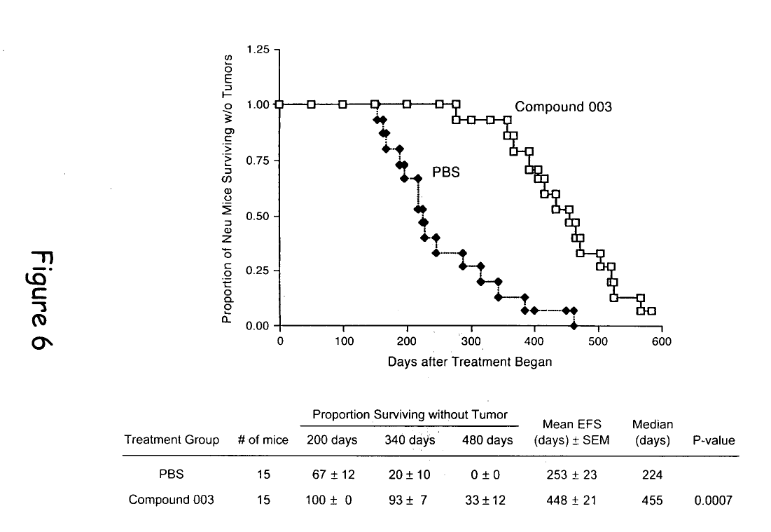

FIG. 7 shows data from a representative MMTV/Neu transgenic mouse with a highly developed mammary tumor. (A) Localization of the mammary-tumor; (B) The excised tumor; (C) H+E staining of a representative section of the mammary gland tumor (D) HER-2 expression on the surface of breast cancer cells from the mammary gland tumor.

FIG. 8 shows a protective effect of Compound 003 in a lactation induced murine model of Her 2 positive breast cancer. MMTV/Neu transgenic mice were treated with Compound 003 that was added to the daily food of the mice at an average daily dose level of 50 mg/kg/day for the duration of the,250-day experiment. In order to maintain the development of mammary gland and maximum expression of the MMTV-driven neu transgene, all female transgenic mice were kept either pregnant or lactating by continued housing with male FVB mice. Mice were screened for the tumor formation every other day. Tumor-free survival was analyzed using life table statistics as previously reported. The protective anti-cancer activity of Compound 003 was evaluated according to the rise of the survival rate and prolongation of the tumor-free survival of the experimental animals as compared with the control animals. Statistical Significance was determined using the Kaplan Meier Log-Rank test.

FIG. 9 is an overlay of macroarrays from Compound 3 and vehicle treated cells. Gene Profiling using the Human Atlas cDNA Expression arrays was preformed as detailed in the Materials and Method section. 588 genes were subdivided into these six functional groups as represented by the following quadrants: (A) Oncogenes, Tumor suppressors Cell Cycle regulators, (B) Ion channels and transport, Modulators, stress response, (C) Apoptosis, DNA synthesis, repair and recombination, (D) Transcription factors, DNA binding proteins, (E) Receptors, Cell surface antigens, & Cell adhesion (F) Growth factors, cytokines, chemokines, Interleukins, Hormones. Shown here is the composite of control and Compound 003 (50TM). The 8 bit grey image of Compound 003 array was overlayed with red color and the control array was overlayed with green color. The superimposition of these two images results in visualization of genes that were increased in expression (red), no change in expression (yellow) and decreased in expression (green) when treated with drug.

FIG. 10 shows changes in gene expression in Compound 003 treated—relative to vehicle—treated cells. Gene expression values were background subtracted, log2 transformed and normalized using the piecewise linear regression method. The control values were plotted against 5 μM (A) and 50 μM Compound 003 (B) treated samples. Genes that showed increase in expression and decrease in expression with both drug concentrations are highlighted as filled black squares.

FIG. 11 shows Table 4 presenting genes significantly affected by Compound 003 treatment.

FIG. 12 shows dose- and time-dependent accumulation of Ala-AZT-MP in CEM (A), BT-20 (B), T98 (C) and Nalm6 cells treated with COMPOUND 003.

FIG. 13 shows inhibition of ala-AZT-MP formation by BNPP, paraoxon, physostigmine, PMSF, in BT20 cells and NALM-6 cells treated with COMPOUND 003.

FIG. 14 shows identification of COMPOUND 003-M1 in vivo. Mass spectra of COMPOUND 003 metabolite (COMPOUND 003-M1) (A) and synthetic COMPOUND 003-M1 (B).

FIG. 15 shows identification of COMPOUND 003-M2 in vivo. Mass spectra of COMPOUND 003 metabolite (COMPOUND 003-M2) (A) and AZT (B); ¹H-NMR of COMPOUND 003 metabolite (COMPOUND 003-M2) (C) and AZT (D))

FIG. 16 shows representative HPLC chromatograms for (A) blank plasma; (B) plasma sample spiked with COMPOUND 003-M1, AZT and COMPOUND 003, and (C) plasma sample at 30 minutes following oral administration of COMPOUND 003.

FIG. 17 graphically shows the stability of COMPOUND 003 in biological fluids. Stability of COMPOUND 003 is shown in plasma (A), in gastric fluid. (B) and in intestinal fluid (C).

FIG. 18 graphically shows pharmacokinetics after intravenous administration of COMPOUND 003. Plasma concentration-time profiles of ala-AZT-MP (A) and AZT (B) in CD-1 mice are shown following intravenous administration of 200 mg/kg COMPOUND 003 (5 mice per time point).

FIG. 19 graphically shows pharmacokinetics after oral administration of COMPOUND 003. Plasma concentration-time profiles of ala-AZT-N4P (A) and AZT (B) in CD-1 mice are shown following oral administration of 200 mg/kg COMPOUND 003 (6 mice per time point).

FIG. 20 shows confocal microscopy of breast cancer cells treated with Compound 003 or AZT. Untreated BT-20 breast cancer cells have microtubules (lighter regions, green) organized throughout the cytoplasm. In contrast, no microtubules are observed in cells treated with the aryl phosphate derivative of AZT, Compound 003 (labeled as DDE003) (185 μM for 24 hours). Similar concentrations of AZT had no effect on microtubules. Lighter areas (green in original color picture) are tubulin. Darker central regions in the lighter areas (blue in original color picture) are DNA. Bar=20 μM

### DETAILED DESCRIPTION OF THE INVENTION

### A. DEFINITIONS

Carboxylesterases, also referred to carboxlic ester hydrolases, aliesterases, EC 3.1.1.1 and CaEs) are hydrolases that split ester bonds. The mechanism follows the general formula: R—C(O)—OX+H2O→R—C(O)—OH+X—OH. There are several known and commercially available carboxylesterase inhibitors, such as paraoxon, diisopropylphosphofluoridate (DFP), Bis(p-nitrophenyl)phosphate (BNPP), and phenylmethyl sulfonyl fluoride (PMSF).

Proliferative cell disorders or hyperproliferative disease refers to cancer and non-cancer proliferative cell disorders. Non-cancer proliferative disorders include, but are not limited to psoriasis, polyps, endometriosis, histrocytosis, mastocytosis, polycytemia, thrombocytosis, auto immune disorders, and certain inflammatory diseases. Proliferative cell disorders may also be characterized by diseases involving invasion and migration of cells into surrounding tissue.

### B. COMPOUNDS OF THE INVENTION

1. Compound 003

“Compound 003” or “AZT phosphoramidate DDE-3”, is 3′-azidothymidine 5′-[p-methoxyphenyl methoxyalaninyl phosphate], an aryl phosphate derivative of AZT. Compound 003 has the following structural formula I:

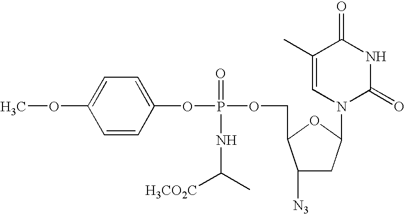

Compound 003 can also be in the form of pharmaceutically acceptable salts, for example, salts formed with organic and inorganic acids. Suitable acids for salt formation with the amino group of the amino acid or amino acid ester residue of Compound 003 include, for example, hydrochloric, sulfuric, phosphoric, acetic, citric, oxalic, malonic, salicylic, malic, gluconic, fumaric, succinic, asorbic, maleic, methanesulfonic, and the like acids. The salts can be prepared, for example, by contacting the free base form of Compound 003 with a sufficient amount of the desired acid to produce a mono-, di-, or greater complexed salt in a conventional manner. Suitable bases for the formation of a salt with the carboxylate group of an amino acid residue of Compound 003 include, for example, sodium hydroxide, sodium carbonate, sodium bicarbonate, potassium hydroxide, potassium carbonate, potassium bicarbonate, and the like bases.

Compound 003 is metabolized in vivo to form alaninyl-AZT-monophosphate (Ala-AZT-MP) as shown below in Scheme 1 illustrating the Metabolic Pathway of Compound 003. AZT=3′-azido-3′-deoxythymidine.

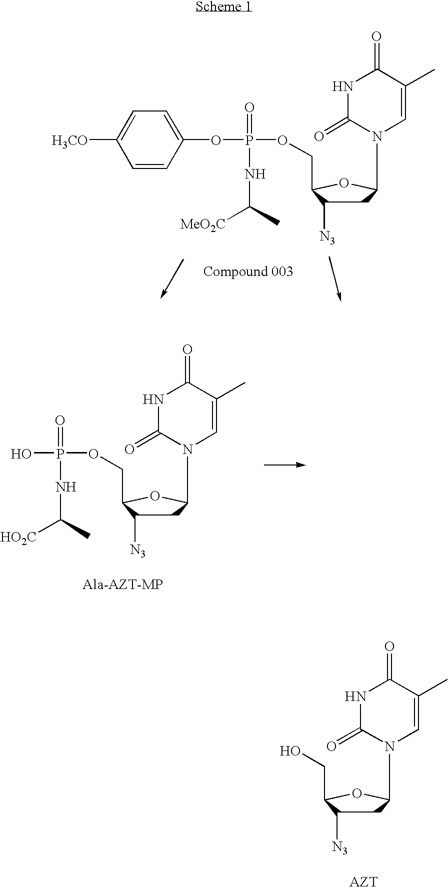

2. Ala-AZT-MP

Ala-AZT-MP (3′-azido-3′-deoxythymidine-5′-alaninyl phosphate) has the following structural formula II:

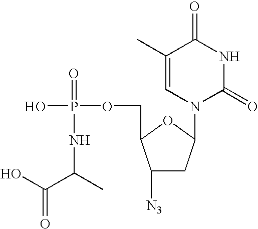

A

la-AZT-MP can also be in the form of pharmaceutically acceptable salts, for example, salts formed with organic and inorganic acids. Suitable acids for salt formation with the amino group of the amino acid or amino acid ester residue of Compound 003 include, for example, hydrochloric, sulfuric, phosphoric, acetic, citric, oxalic, malonic, salicylic, malic, gluconic, fumaric, succinic, asorbic, maleic, methanesulfonic, and the like acids. The salts can be prepared, for example, by contacting the free base form of Compound 003 with a sufficient amount of the desired acid to produce a mono-, di-, or greater complexed salt in a conventional manner. Suitable bases for the formation of a salt with the carboxylate group of an amino acid residue of Compound 003 include, for example, sodium hydroxide, sodium carbonate, sodium bicarbonate, potassium hydroxide, potassium carbonate, potassium bicarbonate, and the like bases.

### C. CARBOXYLESTERASE INHIBITORS

As shown below, carboxylesterases have now been found to mediate conversion of Compound 003 in vivo into alaninyl-AZT-monophosphate (Ala-AZT-MP). Our studies suggest coadministration a carboxylesterase inhibitor with Compound 003 increases the anti-cancer activity of Compound 003, in vivo and in vitro (see Example 2 below). Inhibitors of carboxylesterase are known, and include, for example, paraoxon.

Furthermore, carboxyesterase likely hydrolyses first the methoxyester of alanine side chain of compound 003 and subsequently the methoxy phenyl group leaves forming the metabolite. Ala-AZT-MP.

### D. ADMINISTRATION METHODS

Compound 003 can be formulated as pharmaceutical compositions and administered to a mammalian subject, including a human patient, in a variety of forms adapted to a chosen route of administration. The compounds are typically administered in combination with a pharmaceutically acceptable carrier, and can be combined with specific delivery agents, including targeting antibodies or cytokines.

The compounds can be administered orally, parentally (including subcutaneous injection, intravenous, intramuscular, intrasternal or infusion techniques), by inhalation spray, topically, by absorption through a mucous membrane, or rectally, in dosage unit formulations containing conventional non-toxic pharmaceutically acceptable carriers, adjuvants or vehicles. Pharmaceutical compositions of the invention can be in the form of suspensions or tablets suitable for oral administration, nasal sprays, creams, sterile injectable preparations, such as sterile injectable aqueous or oleagenous suspensions or suppositories.

For oral administration as a suspension, the compositions can be prepared according to techniques well-known in the art of pharmaceutical formulation. The compositions can contain microcrystalline cellulose for imparting bulk, alginic acid or sodium alginate as a suspending agent, methylcellulose as a viscosity enhancer, and sweeteners or flavoring agents. As immediate release tablets, the compositions can contain microcrystalline cellulose, starch, magnesium stearate, and lactose or other excipients, binders, extenders, disintegrants, diluents, and lubricants known in the art.

For administration by inhalation or aerosol, the compositions can be prepared according to techniques well-known in the art of pharmaceutical formulation. The compositions can be prepared as solutions in saline, using benzyl alcohol, or other suitable preservatives, absorption promoters to enhance bioavailability, fluorocarbons or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

For rectal administration as suppositories, the compositions can be prepared by mixing with a suitable non-irritating excipient, such as cocoa butter, synthetic glyceride esters or polyethylene glycols, which are solid at ambient temperatures, but liquefy or dissolve in the rectal cavity to release the drug.

Solutions or suspensions of the compounds can be prepared, for example, in water, isotonic saline (PBS), and optionally, mixed with a nontoxic surfactant. Dispersions may also be prepared, for example, in glycerol, liquid polyethylene, glycols, DNA, vegetable oils, triacetin, and mixtures thereof. Under ordinary conditions of storage and use, these preparations may contain a preservative to prevent the growth of microorganisms.

A pharmaceutical dosage form suitable for injection or infusion use can include, for example, sterile, aqueous solutions or dispersions, or sterile powders comprising an active ingredient, that is adapted for the extemporaneous preparation of sterile injectable or infusible solutions or dispersions. The ultimate dosage form should be sterile, fluid, and stable under conditions of manufacture and storage. A liquid carrier or vehicle can be a solvent or liquid dispersion medium comprising, for example, water, ethanol, a polyol such as glycerol, propylene glycol, or liquid polyethylene glycols, and the like, vegetable oils, nontoxic glyceryl esters, and suitable mixtures thereof. The proper fluidity can be maintained, for example, by the formation of liposomes, by the maintenance of-the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it may be desirable to include isotonic agents, for example, sugars, buffers, or sodium chloride. Prolonged absorption of the injectable compositions can be brought about by inclusion in the composition of agents delaying absorption—for example, aluminum monosterate hydrogels and gelatin.

Sterile injectable solutions are prepared by incorporating the compound to be administered in the required amount in the appropriate solvent with various other ingredients as enumerated above and, as needed, sterilized, for example, by filter sterilization. In the case of sterile powders for the preparation of sterile injectable solutions, preferred methods of preparation include vacuum drying and freeze-drying techniques that yield a powder of the active ingredient.

### E. INHIBITORY METHODS

Cell Cycle Arrest

In diseases of excessive cell proliferation, for example cancer, the inhibition of cell division is of interest. Eukaryotic cell growth, and division is described by the following phases: G₁ (first gap) phase, S (synthetic) phase, G₂ (second gap) phase and M (mitotic) phase. G₁, S, and G₂ are collectively referred to as “interphase” during which DNA and other cellular macromolecules are synthesized. During M phase, the nucleus and cell physically divide.

Cell Division relies on microtubules which are built, during G₂/M into a radial array (bipolar mitotic spindle) that segregates the sister chromatids into each daughter cell. The precisely regulated dynamic rearrangements of the microtubules make them one target for drugs that interfere with cell cycle progression. (See, Miyamoto, et al., 2003, Progress in Cell Cycle Res., 5:349). Arrest of the cell cycle, for example by interfering with action of microtubules, can be seen in a variety of effects, including inhibiting mitotic spindle formation, aster growth and G₂ arrest.

1. Inhibiting Mitotic Spindle Formation

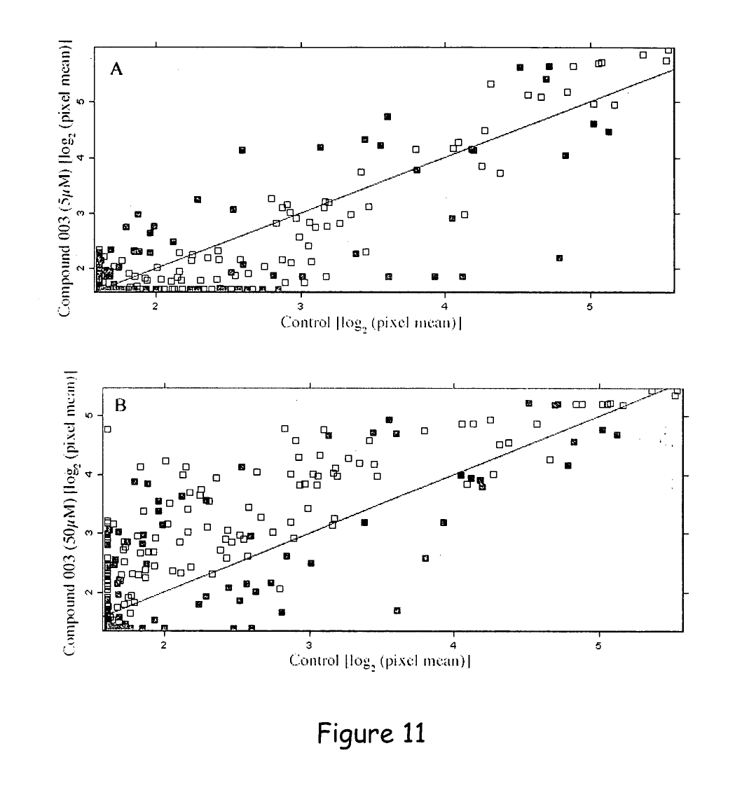

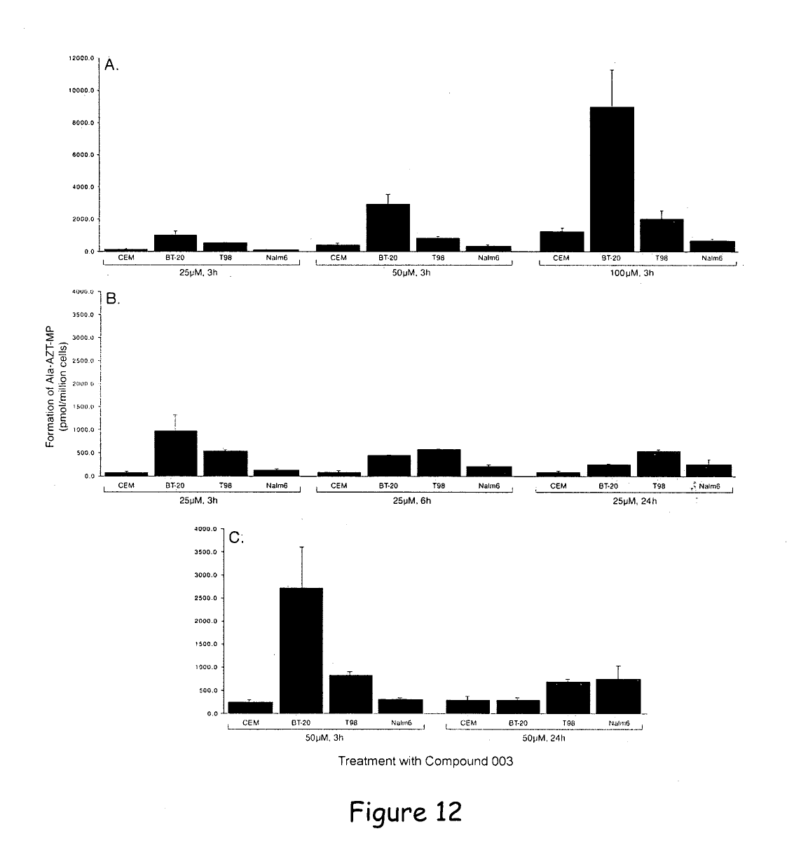

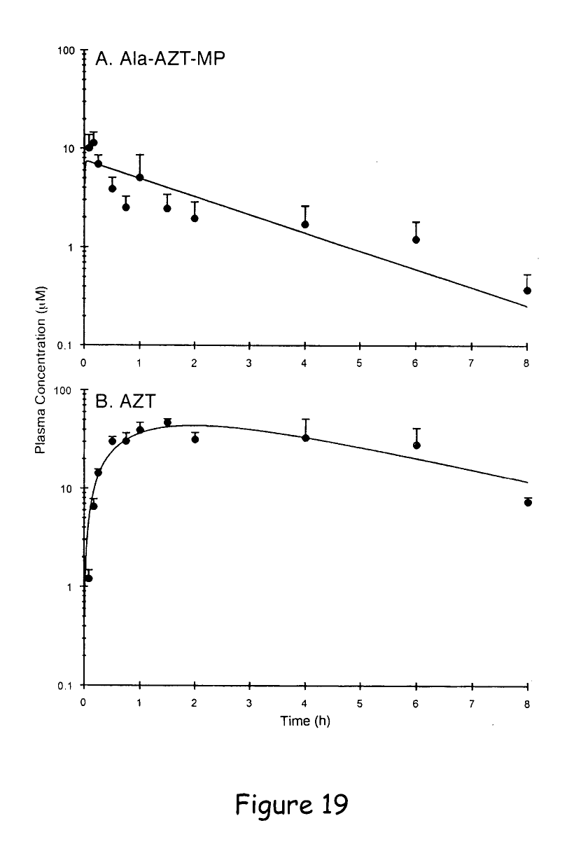

Compound 003 was initially screened for microtubule interference activity using an in vitro centrosomal regrowth assays. A centrosome is a center of microtubule organization during the division of a nucleus. Compound 003 inhibited nucleation of microtubules and subsequent aster growth from isolated centrosomes in in vitro centrosomal regrowth assays. Confocal laser scanning microscopy experiments further demonstrated that Compound 003 prevents normal microtubule assembly and initiates aberrrant multipolar spindle formation in intact human cancer cells. See Example 1 and FIGS. 11 and 12. In contrast, AZT had no effect on microtubules in BT20 cells (FIG. 19).

2. Initiating G2 Arrest

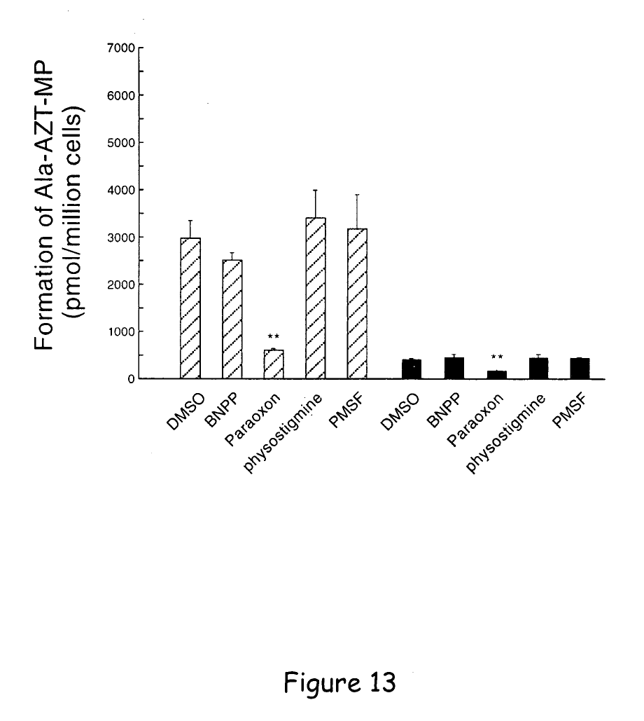

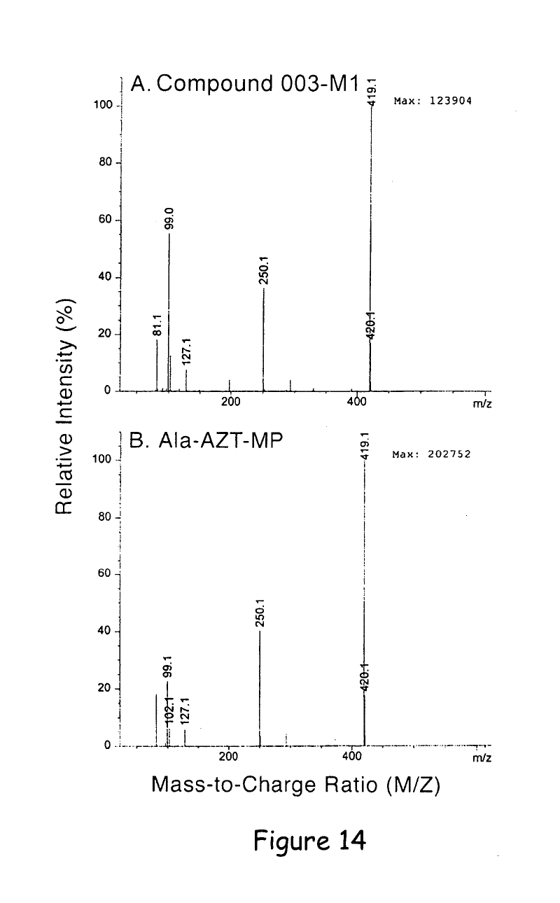

Activity of Compound 003 to initiate cell cycle arrest was also determined in vivo in a variety of human cancer cells. DNA flow cytometry analysis of five human cancer cell lines and an aggressive chicken B lymphoma (DT40) cell line treated with Compound,. 003 all showed a marked increase in the proportion of nuclei in the G₂M peak of DNA;. histograms with a concomitant decrease in the proportion of nuclei in the G_{0/1} peak. See Example 1 and FIGS. 13 and 14. These results show Compound 003 causes cell cycle arrest in a variety of human cancer cells and chicken B lymphoma.

These results indicate the Compound 003 is useful as an agent to initiate cell cycle arrest in actively dividing cells, including cancer cells. These results further support Compound 003's utility as an anti-cancer agent.

F. Methods of Treatment for Proliferative Cell Disorders Treating Cancer

1. Toxicity

Effective drugs for cancer treatment are toxic to cancer cells, and are preferably less toxic to non-cancerous (i.e. normal) cells. This concept can also be described by a “therapeutic window” referring to an allowed range of therapeutic dosages for a particular drug. For an anti-cancer drug, the minimum amount that is administered is a therapeutically effective amount, that is the amount that results in the desired anti-cancer effects (e.g., cell cycle arrest in cancer cells). The maximum allowed dose is determined by the acute toxic effects on normal cells or subject being treated, also referred to as a safety profile.

Compound 003 was tested for toxicity in CD-1 mice and Lewis rats. Compound 003 was found to be non-toxic in CD-1 mice at doses up to and including 80 mg/kg under the study conditions described in Example 1. Compound 003 is also well tolerated by Lewis rats at daily dose levels as high as 80 mg/kg and cumulative dose levels as high as 2.4 g/kg (Also desribed in Example 1). These findings suggest that Compound 003 has a sufficiently high maximum allowed dose without notable toxic effects so as to be suitable for use in the treatment of cancer.

2. Anti-Cancer Activity

The effect of Compound 003 on spontaneous breast cancer development was examined in MMTV/Neu Transgenic mice, serving as a model for human breast cancer. One group of transgenic mice Were treated with Compound 003 at a nontoxic dose level of 50 mg/kg/day, while a second group served as a control. Development of breast cancer was delayed in the Neu transgenic mice treated with Compound 003. Compound 003 prolonged the median cancer-free survival from 224 days (control group) to 455 days for the treated mice. See Example 1. These results demonstrate that Compound 003 has in vivo anti-cancer activity in the MMTV/neu transgenic mouse model of breast cancer.

### EXAMPLES

The invention may be better understood with reference to the following Examples.

These examples are meant to exemplify specific embodiments of the invention, and are not intended to limit the invention in any way.

### Example 1

### Anti-Cancer Activity Profile of Compound 003

1.1 Introduction

The purpose of this study was to examine the in vitro and in vivo anti-cancer activity as well as in vivo toxicity profile of the zidovudine derivative 3′-Azidothymidine 5′-[p-Methoxyphenyl methoxyalaninyl phosphate] (“Compound 003”), which was identified as a candidate anti-breast cancer agent because of its in vitro activity in a functional assay of isolated centrosomal. Compound 003 prevented bipolar mitotic spindle assembly and caused a G2 arrest in human cancer cells. Compound 003 was very well tolerated by both mice and rats without any toxicity at cumulative dose levels >2 g/kg. Notably, Compound 003 prolonged cancer-free survival in the MMTVneu transgenic, mouse model of HER2 positive breast cancer. The remarkable in vivo activity and safety profile of Compound 003 suggests use of this promising new anti-cancer agent for clinical use in breast cancer patients.

1.2 Materials and Methods

1.2.1 Chemicals

All the reagents used in this study were HPLC grade. Deionized water was prepared via a Milli-Q purification system (Medford, Mass.). Acetonitrile was purchased from Burdick & Jackson (Allied Signal Inc., Muskegon, Mich.). Methanol, acetic acid and hydrochloric acid were purchased from Fisher Chemicals (Fair Lawn, N.J.). Ammonium phosphate, phosphoric acid and AZT were purchased from Sigma (St. Louis, Mo.).

1.2.2 Synthesis and Characterization of Compound 003

3′-Azidothymidine 5′-[p-Methoxyphenyl methoxyalaninyl phosphate] (“Compound 003”) was prepared according to the literature procedure described in: McGuigan et al., 1993, J Med Chem. 36: 1048; McGuigan et al., 1996, J Med Chem. 39:1748; and Egron, et al., 1998, Bioorg Med Chem Lett. 8:1045. The synthesis started with a condensation reaction of p-methoxyphenol with phosphorus oxychloride, generating phosphorodichloridate (1 in Scheme 2). Intermediate 1 in turn condensed with alanine methyl ester, furnishing phosphorochloridate 2. Condensation of 2 with AZT afforded the aryl phosphoramidate derivative of AZT (Compound 003). Because of the tetrahedral configuration at the phosphorus center, the reaction product is a mixture of two diastereomers.

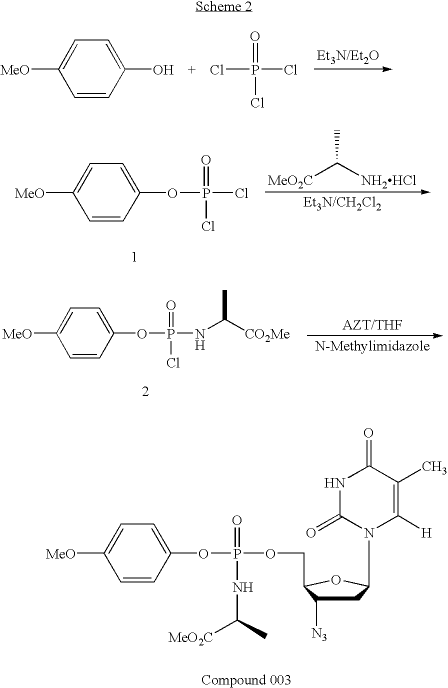

Analytical thin-layer chromatography (TLC) was performed on Merck pre-coated glass plates (silica gel 60, F₂₅₄, 250-μm thickness), and visualized under 254-nm UV light. The preparative column chromatography was performed using EM silica gel 60, 230-400 mesh. NMR spectra were recorded on a Varian 300, using CDCl₃ with tetramethylsilane as the internal standard for ¹H (300 MHz), solvent as the internal standard for ¹³C (75 MHz), or phosphoric acid as the external standard for ³¹P (121 MHz). Mass spectra (MALDI-TOF) were recorded by a G2025A LD-TOF system (Hewlett Packard). MS samples were prepared with 1:1 =matrix (30-mM alpha-cyano-4-hydroxycinnamic acid in 50%: 30%: 20%=acetonitrile:methanol:water): NMR sample in CDCl₃. HPLC data was recorded using an HP 1100 system, with a Zorbax SB-C3 column (5 μm, 4.6×150 mm), with water (50 mM NH₄H₂PO₄/H₃PO₄, pH 2.5): acetonitrile=70:30 with a flow rate of 1 mL/min and detection by UV at 218 nm. Reactions were performed in glassware which had been oven dried (120° C./overnight), and under nitrogen atmosphere. All reaction mixtures were stirred magnetically, unless otherwise noted. Solvents and reagents were used as purchased from Aldrich Chemical Co., unless otherwise stated. The yields quoted in this paper were isolated yields. Compound 003 was identified by NMR, GC/MS, IR and UV spectrometry. The purity was further confirmed by thin layer chromatographic technique (TLC) and HPLC and was found to be over 95.0%.

1.2.3 Characteristics of 3′-Azidothymidine 5′-[p-Methoxyphenyl methoxyalaninyl phosphate] (Compound 003).

¹H NMR (CDCl₃. Starred peaks are split due to diastereoisomers.) δ8.35 (s, 1H), 7.37 (t, J=1.5 Hz, 1H), 7.15-7.10 (m, 2H), 6.85-6.82 (m, 2H), 6.21 (t, J=6.6 Hz, 2/3H), 6.15 (t, J=9.0 Hz, 1/3H), 4.37-4.33 (m, 2H), 4.07-4.01 (m, 1H), 3.78* (s, 3H), 3.73 (s, 1H), 3.72 (s, 2H), 3.63 (t, J=11.1 Hz, 1H), 2.42-2.34 (m, 1H), 2.24-2.14 (m, 1H), 1.91 (d, J=1.2 Hz, 2H), 1.90 (d, J=0.9 Hz, 1H), 1.39 (d, J=7.5 Hz, 1H), 1.36 (d, J=6.9 Hz, 2H); ¹³C NMR (CDCl₃) δ 173.9, 173.7, 163.6, 1.56.7, 150.1, 143.7, 143.6, 135.2, 120.9, 120.8, 111.3, 107.2, 85.0, 84.7, 82.3, 82.2, 82.1, 77.2, 65.7, 65.6, 65.5, 60.4, 60.3, 55.6, 52.6, 50.4, 50.2, 37.3, 21.0, 20.9, 12.5; ³¹P NMR (CDCl₃) 3.3, 3.0; MALDI-TOF MS m/z 539.0 (MH⁺), 56.1.1 (MNa⁺); HPLC retention time 10.8 (50%), 11.2 (50%) min; UV λmax 192, 216, 268 nm.

1.2.4 Tumor Cell Lines

The following tumor cell lines were used in the described experiments: BT-20, human breast cancer cell line; DU-145, human prostate cancer cell line; U373, human glioblastoma cell line; MOLT-3, human T lymphoma cell line; DAUDI, human Burkitt's lymphoma cell line; DT40, chicken B lymphoma cell line; RS4; 11, human infant leukemia cell line; NALM-6, human pre-B acute lymphoblastic leukemia cell line; JURKAT, human T leukemia/lymphoma cell line; and PC-3, human prostate adenocarcinoma cell line.

1.2.5 Isolation of Centrosomes and Functional Assay of Aster Formation

Centrosome isolation from an EBV-transformed B-lymphoblastoid cell line was performed following a slight modification of a literature procedure (See, for example, Moudjou and Bomens, 1994, Cell Biology: A Laboratory Handbook, p. 595. Academic Press, Inc, New York; and Cell Biology: A Laboratory Handbook, 1994).

Briefly, cells were grown to a density of 1×10⁶ per ml. A total of 1×10⁹ cells were treated for 1 hour with 0.2 mM nocodazole and 1 mg/ml Cytochalasin D. All subsequent procedures were performed at 4° C. Firstly, cells were transferred into sterile conical 50 ml tubes and sedimented at 1,200×g for 5 minutes. Cells were then washed twice with 50 ml of filtered tris buffered saline (10 mM Tris-HCl, pH 7.4, 150 mM NaCl, TBS) by gentle resuspension with a pasteur pipette and sedimented at 1,200×g for 5 minutes. Cells were next washed with 8% sucrose in ten times diluted TBS and sedimented at 1,200×g for 5 minutes. Cells were lysed with 100 ml of 1 mM Hepes lysis buffer (0.5% Nonidet-P40), 0.5mM MgCl2, 0.1% β-Mercaptoethanol, 1 mM PMSF, 100 μg/ml aprotinin, 1 μg/ml leupeptin and 1 μg/ml pepstatin), pH 7.2. The swollen nuclei were sedimented at 1,200×g for 10 minutes and the supernatant was filtered through a 70 μm nylon cell strainer. After the addition of 1M Hepes (10 mM final) and 5 μl of 10 mg/ml DNase (1 μg/ml final), the lysate was incubated for 30 minutes at 4° C. 2.5 ml of a 60% sucrose solution was placed at the bottom of four 30 ml SW28 tubes. 25 ml of the lysate was overlayed and the centrosomes were sedimented into this cushion at 10,000×g for 30 minutes. The supernatant was then removed until 7 ml remained in the bottom of each tube. The centrosomal solution was then vortexed vigorously and overlayed on a discontinous sucrose gradient (5ml of 70%/0 sucrose, 3 ml of 50% sucrose, 3 ml of 40% sucrose and spun at 40,000×g for 1 hour at 4° C. Fourteen 0.5 ml density gradient fractions were collected. An aliquot was saved for Western blot analysis and confocal microscopy. Remaining samples were immediately frozen in liquid N₂.

To determine if Compound 003 would inhibit the nucleation of microtubules and subsequent aster formation, centrosomal regrowth from isolated centrosomes was accomplished following a slight modification of a literature procedure (See, for example, Moudjou and Bornens, 1994, Cell Biology: A Laboratory Handbook, p. 595. Academic Press, Inc, New York; and Cell Biology: A Laboratory Handbook, 1994).

Briefly, a solution containing 50 μl purified tubulin (2.5 mg/ml), ⁵ μl isolated centrosomes (5×10 5 centrosomes) and 10 μl RG1 buffer (80 mM Pipes, pH 6.8, 1 mM MgCl2, 1 mM EGTA, 1 mM GTP) was incubated at 37° C. for 8 minutes in an eppendorf tube. Following the addition of 200 μl 1% glutaraldehyde (in RG1 buffer), the tube was placed at 25° C. for 3 minutes and then placed on ice. 1 ml ice cold RG2 buffer (RG1 buffer without GTP) was added to this tube. 5 mls of 25% glycerol (in RG2 buffer) Was added each 15 ml Corex tube with corresponding adapters and glass coverslips. This solution was overlayed with the nucleated microtubule sample and the asters were sedimented at 20,000×g for 10 minutes with a JS 13.1 rotor in a Beckman Centrifuge at 4° C. Following the spin, 1 ml of the solution was aspirated from the top of the tube and replaced with 1 ml of 1% Tx-100 solution. All of the 5 mls of glycerol were removed, leaving 1 ml of the Tx-100 solution over the coverslips and recovering the latter from the Corex tube.

1.2.6 Analysis of Compound 003-induced Mitotic Arrest Using DNA Flow Cytometry

Exponentially growing tumor cells were cultured at 1×10⁶ cells/ml in clonogenic medium (RPMI 1640 medium +1% penicillin/streptomycin +10% heat-inactivated fetal bovine serum, 2 mML-glutamine, and 10 mM Hepes buffer) in the presence or absence of 100 μM compound 003 for 48 hour at 37° C., 5% CO2. At 48 h, cells were washed two times in fresh clonogenic medium, trypsinized, fixed in ice cold ethanol and labeled with propidium iodide to quantify their DNA content, as described previously (See, for example, Navara, et al., 2001, Anticancer Drugs. 12:369). Quantitative DNA analysis was performed on a Becton Dickenson Calibur flow cytometer equipped with a Consort 40 computer using the COTFIT program, which includes CELLCY, a cell cycle distribution function that fits DNA content histograms and calculates-the percentages of cells in G, S, and GM phases of the cell cycle, as described, for example, in Uckun et al., 1996, J Biol Chem. 271, 6389.

1.2.7 Confocal Laser Scanning Microscopy

Immunofluorenscence was used to examine the spindle features of human cancer cell lines treated with Compound 003 or 0.5% DMSO. Initially, cells at log phase were seeded onto sterile 22 mm coverslips in six -well culture plates. 24 hours later, the cells were treated with compound 003 for 2 hours at 37° C., 5% C02, fixed in methanol (−20° C. 15 min) followed by a 15 minute incubation with PBS +0.1.% Tx-100. The coverslips were incubated with antibodies raised against α-tubulin, γ-tubulin (Sigma Chemical Co., St Louis, Mo.) (40 min 37° C.), washed in PBS-Tx-100, and incubated with an appropriate secondary antibody conjugated to FITC (Jackson ImmunoResearch, Westgrove, Pa.). Cellular DNA was labeled with 5 μM TOTO3 (Molecular Probes, Eugene, Oreg.) for 20 minutes. Coverslips were immediately inverted onto slides in Vectashield (Vector Labs, Burlingame, Calif.) to prevent photobleaching, sealed with nail varnish and stored at −20° C. The slides were examined using a Bio-Rad MRC-1024 Laser Scanning Confocal Microscope equipped with a Kr/Ar laser (BioRad, Hercules, Calif.) mounted on a Nikon Eclipse E800 upright microscope with high numerical aperture objectives (Nikon, Melville, N.Y.). Digital data were processed using Lasersharp (BioRad) and Adobe Photoshop software (Adobe Systems, Mountain View, Calif.). ( See, for example, Navara, et al., 2001, Anticancer Drugs. 12:369; Malaviya, et al., 2002, Leuk Lymphoma. 43, 1329; and Ghosh, 1998, Clin Cancer Res. 4, 2657).

1.2.8 Animals Female CD-1 mice (Body weight: ˜25 g; Age: 7 week old) were purchased, from Charles River Laboratories (Willmington, Mass., USA). MMTV Neu-mice [FVB/N-TgN(MMTV neu)202MUL; Jackson Laboratory, Bar Harbor, Me.] were bred to produce multiple litters. All mice were housed in microisolator cages (Lab Products, Inc., Maywood, N.Y., USA) containing autoclaved bedding in a controlled specific pathogen-free (SPF) environment (12-h light/12-h dark photoperiod, 22±1° C., 60±10% relative humidity), which is fully accredited by the USDA (United States Department of Agriculture). Adult male Lewis rats (Body weight: ˜160 g) were obtained from the specific pathogen-free (SPF) breeding facilities of Harlan Sprague Dawley (Indianapolis, Id., USA) at 14 weeks of age. All husbandry and experimental contact made with the mice and rats maintained SPF conditions. The rats were kept in microisolater cages (Allentown Caging Equipment Co., Inc., Allentown, N.J., USA) containing autoclaved food, water, and bedding. Animal studies were approved by Parker Hughes Institute Animal Care and Use Committee and all animal care procedures 1.5 conformed to the Guide for the Care and Use of Laboratory Animals (National Research Council, National Academy Press, Washington D.C. 1996, USA).

1.2.9 Toxicity Studies of Compound 003 in Mice and Rats.

The toxicity profile of Compound 003 in CD-1 mice and Lewis rats was examined, using standard procedures as previously reported for other experimental drugs (Uckun, et al., 2003, Arzneimittelforschun. 53:357; Uckun, et al., 2002, Clin Cancer Res. 8: 1224; Uckun, et al., 2002, Antimicrob Agents Chemother. 46:3428). In brief, mice and rats were treated with intraperitoneal injections of vehicle or Compound 003 at multiple dose levels. Compound 003 was administered as a 0.2 mL (for mice) or 0.5 mL (for rats) bolus injection containing 10% DMSO as a vehicle. Animals were allowed free access to autoclaved standard pellet food and tap water throughout the experiments and monitored daily for morbidity and mortality. Animals were electively sacrificed on day 7 or day 30 to determine the toxicity of Compound 003 by examining their blood chemistry profiles, blood counts, and evaluating multiple organs for the presence of toxic lesions. Blood was collected by intracardiac puncture following anesthesia with ketamine:xylazine and immediately heparinized. The blood chemistry profiles were examined using a Synchron CX5CE Chemical Analyzer (Beckman Instruments, Inc., Fullerton, Calif., USA). Blood counts (red blood cells [RBC], white blood cells [WBC] and platelets [Plt]) were determined using a HESKA Vet ABC-Diff Hematology Analyzer (HESKA Corporation, Fort Collins, Colo., USA). Absolute neutrophil counts (ANC) and absolute lymphocyte counts (ALC) were calculated from WBC values after determining the percentages of neutrophils and lymphocytes by a manual differential count. At the time of necropsy, 22 different tissues from mice (bone, bone marrow, brain, cecum, heart, kidney, large intestine, liver, lung, lymph node, ovary, pancreas, skeletal muscle, skin, small intestine, spleen, stomach, thymus, thyroid gland, urinary bladder, and uterus, as available) and 21 different tissues from rats (bone, bone marrow, brain, coagulating gland, epididymis, heart, large intestine, small intestine, kidney, liver, lung, pancreas, peripheral nerve/spinal nerve, skeletal muscle, skin, spinal cord, spleen, stomach, testes, thymus, urinary bladder) were collected within 15 minutes after sacrifice, gross pathological findings were documented, organs were preserved in 10% neutral phosphate buffered formalin, and processed for histopathological examination. For histopathologic studies, formalin fixed tissues were dehydrated and embedded in paraffin by routine methods. Glass slides with affixed 4-5 micron tissue sections were prepared and stained with Hemotoxylin and Eosin (H&E).

1.2.10 Murine Model of Her2 Positive Breast Cancer

Overexpression of the wild type Neu in the mammary glands of transgenic mice induces metastatic breast cancer. Accordingly, in the MMTV/Neu transgenic strain, the wild-type neu is overexpressed in the mammary gland under the control of the MMTV long terminal repeat. This animal model has been used to analyze the Her2/neu overexpression in breast epithelia, as well as the efficacy of new therapeutic approaches to prevent or treat Her2/neu overexpressing malignancies.

The genotype of mice was confirmed by multiplex polymerase chain reaction (PCR) tests. In brief, a 0.5 inch (1.27 cm) tail tissue section was taken from each mouse and digested at 55° C. in 600 μL lysis buffer (50 mM Tris pH 8.0, 100 mM EDTA, 100 mM NaCl, 1% SDS) with 50 μL Proteinase K (10 mg/mL). Genomic DNA was purified with phenol and chloroform extractions and ethanol precipitation. See, for example, Treco, D. A., 5th, p. 2.1.1. John Wiley and Sons, New York (1995).

Four primers were employed in the PCR tests: a 30-base primer Neu R (5′-CCG GGC AGC CAG GTC CCT GTG TAC AAG CCG-3′)(SEQ ID NO: 1), a 25-base primer Neu F (5′-GGA AGT ACC CGG ATG AGG GCA TAT G-3′) (SEQ ID NO:2), a 21-base primer BcaseinF (5′-GAT GTG CTG CAG GCT AAA GTT-3′)(SEQ ID NO:3) and a 21-base primer BcaseinR (5′-AGA AAC GGA ATG TTG TGG AGT-3′)(SEQ ID NO:4) (Biosynthesis, Lewisville, TX-100). The NeuR×NeuF PCR primer pair yielded a 700 bp PCR product in tissues from mmtv Neu mice. The BcaseinF x BcaseinR primer pair yielded a 500 bp PCR product as the internal positive control. Each 100 μL PCR reaction medium consisted of 1× PCR buffer II containing 2.5 mM MgCl₂ (Perkin Elmer's Amplitaq Gold Kit), 0.2 mM dNTP, (Boehringer Mannheim), 0.4 μM of each, primer and 2.5 U AmpliTaq Gold enzyme. The PCR conditions were 95° C. for 5 minutes, 94° C. for 1 minutes, 30 cycles [60° C. for 2 minutes, 72° C. for 3 minutes], 72° C. for 7 minutes, then 4° C. hold (Touchdown, Hybaid, Potomac, Md.). The product was ran on a standard 1% agarose gel and bands were visualized under UV light using the Eagle Eye II Digital Imager (Stratagene) (Treco, 1995, Supra).

In order to confirm HER-2 expression in mammary tumors, animals were euthanized, and tumors were embedded in paraffin blocks for histological analysis. For histopathologic studies, tissues were fixed in 10% neutral buffered formalin, dehydrated, and embedded in paraffin by routine methods. Glass slides with affixed 4-5 micron tissue sections were prepared and stained with Hemotoxylin and Eosin (H&E) or DAKO HercepTest™. The immunocytochemical analysis utilizing the DAKO HercepTest™ kit (DAKO Corporation,.Carpintera, Calif.) was performed according to the manufacturer's specifications.

MMTV/Neu transgenic mice were treated with Compound 003 that was added to the daily food of the mice at an average daily dose level of 50 mg/kg/day for the duration of the experiment. Mice were screened for the tumor formation every other day. Tumor-free survival was analyzed using life table statistics as previously reported. The protective anti-cancer activity of Compound 003 was evaluated according to the rise of the survival rate and prolongation of the tumor-free survival of the experimental animals as compared with the control animals. Statistical Significance was determined using the Kaplan Meier Log-Rank test.

1.2.11 Gene Profiling: Atlas Human cDNA Expression Arrays

Gene Profiling using the Atlas Human cDNA Expression arrays from Molecular Dynamics was preformed according to the manufacturer's specification, as described below.

Total RNA was prepared from NALM6 cells treated with Compound 003 (5, 50TM, vehicle) using Tripure isolation reagent (Boehringer Mannheim); the reagent is a monophasic solution of phenol and guanidine thiocyanate, followed by Dnase treatment (RQ1 Rnase-Free Dnase, Promega). Total RNA was quantified using standard spectrophotometric methods.

For probe preparation and hybridization, approximately 5 micrograms of total RNA was converted into radioactively labeled cDNA using [γ-32P] dATP (Amersham) by reverse transcriptase (Clontech). Chroma Spin columns (Clontech) were used to purify labeled cDNA. Atlas cDNA probe was denatured with 1M NaOH/10 mM EDTA and neutralized with 1M NaH₂PO₄ and C₀ₜ₋₁ DNA at 68° C. Prehybridization solution was discarded and arrays were hybridized overnight. Arrrays were washed 4 times by using 2×SSC/1% SDS and 2 times by using 0.1×SSC/0.5% SDS. Membranes were sealed and exposed to a phosphoscreen (Molecular Dynamics). The expression levels were quantified using 8 bit gray levels using image analysis software (Imagene 4.2, Biodiscovery; Inc).

For the Atlas Human cDNA Expression array, 588 genes were subdivided into these six functional groups: (A) Oncogenes, Tumor suppressors Cell Cycle regulators, (B) Ion channels and transport, Modulators, stress response, (C) Apoptosis, DNA synthesis, repair and recombination, (D) Transcription factors, DNA binding proteins, (E) Receptors, Cell surface antigens, Cell adhesion & (F) Growth factors, cytokines, chemokines, Interleukins, Hormones. The letters correspond to individual quadrants on the array and may be noted as the first letter in each abbreviation in Table 4.

Spots on the macro arrays (5, 50 Tm and control) were analyzed using the spot finder technique in the Imagene software. The integrity and fragmentation: due to saturated pixels, of the spots was assessed and appropriate background measurements were chosen to detect signals. Subtracting the median value of the background pixels of all the spots in each sub-grid region resulted in the estimation of the background value in that region (i.e regions A to F on the array). All drug treated samples (5, 50TM) were compared to no drug controls using a linear regression method for log2 transformed density readings (Genesight 3.1, Biodiscovery). A piecewise linear regression method (3 bins, minimum of 20 elements per bin) was used normalize density readings across the three arrays to minimize the effect of low expression and saturated signals on the regression. All housekeeping genes were monitored for detection of false positive signals.

1.3 Results

1.3.1 In vitro Activity Profile of Compound 003

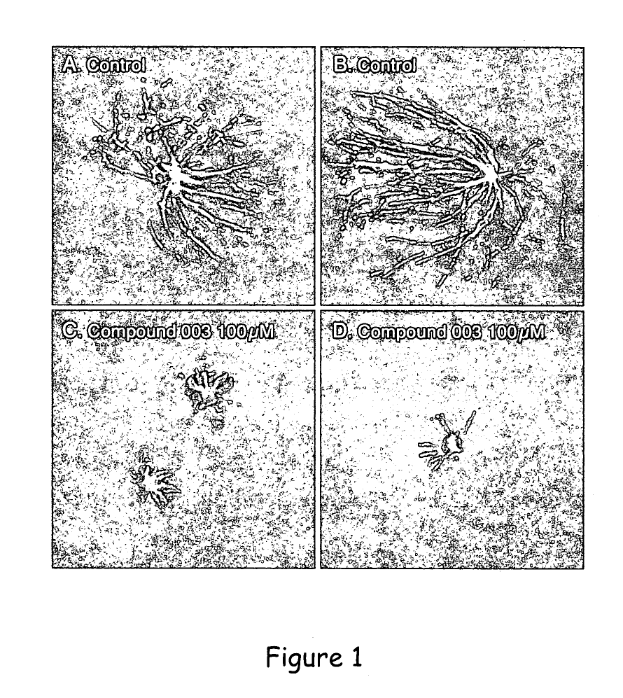

In vitro centrosomal regrowth assays was used as a screening tool to identify novel anti-cancer agents capable of disrupting microtubule assembly. The organization and size of asters from centrosomes isolated from an EBV-transformed B-lymphoblastoid cell line treated with various compounds were compared. Compound 003 was identified as a candidate agent because it inhibited nucleation of microtubules and subsequent aster growth from isolated centrosomes. The effect of Compound 003 on aster formation in isolated centrosomes is shown in FIG. 1. The presence of Compound 003 inhibited the ability of the centrosomes to nucleate and sufficiently grow microtubule asters. Two representative asters from each experiment are shown. Cells illustrated in FIG. 1 A&B were treated with vehicle only. FIG. 1 C&D were treated with Compound 003 100 μM.

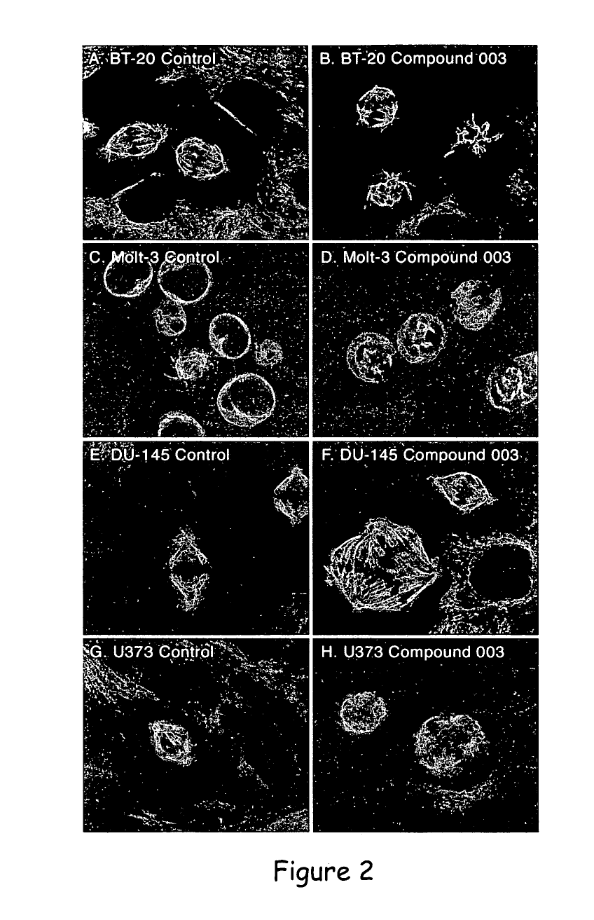

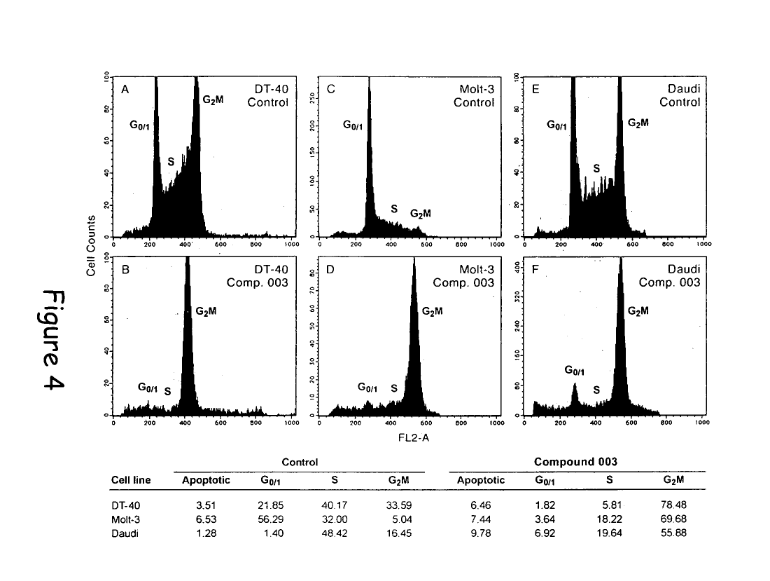

Confocal laser scanning microscopy experiments demonstrated that Compound 003 prevents the normal process of microtubule assembly and induced aberrant multipolar (instead of normal bipolar) spindle formation in intact human cancer cells, as illustrated with BT20 breast cancer cells, MOLT-3 T-lymphoma cells, DU145 prostate cancer cells, U373 glioblastoma cells and PC-3 prostate cancer cells and MDA-MB breast cancer cells (FIGS. 2 & 3). The ability of Compound 003 to cause cell cycle arrest in human cancer cells was confirmed using DNA flow cytometry. Compound 003 caused G2 arrest in five of five human cancer cell lines and an aggressive chicken B lymphoma (DT40) cell line, as reflected by a marked increase of the proportion of nuclei in the G2M peak of the DNA histograms with a concomitant decrease of the proportion of nuclei in the G0/1 peak (FIGS. 4 & 5).

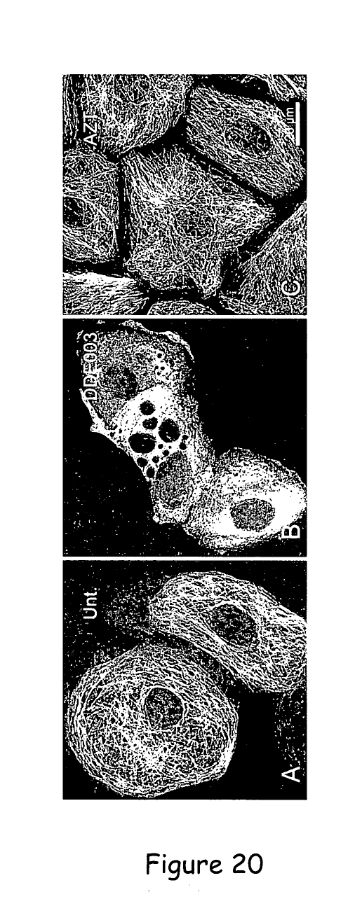

Confocal microscopy of breast cancer cells treated with Compound 003 or AZT is shown in FIG. 20. Untreated BT-20 breast cancer cells show microtubules (light regions green in original) organized throughout the cytoplasm. In contrast, no microtubules were observed in cells treated with the aryl phophate derivative of AZT, Compound 003 (labeled as DDE003) (185 μM for 24 hours). Similar concentrations of AZT had no effect on microtubules. Lighter areas (green in original color picture) are tubulin. Darker central regions in the lighter areas (blue in original color picture) are DNA. Bar=20 μM.

1.3.2 Toxicity of Compound 003 in CD-1 Mice.

We first examined the acute toxicity profile of Compound 003 when it was administered in a single intraperitoneal bolus injection. Results are shown in Table 1&2 below.

Two-hundred 7-week old female CD-1 mice were treated with a single intraperitoneal bolus dose of Compound 003 in 0.2 mL 10% DMSO/PBS at dose levels of 10 mg/kg/dose (n=40), 20 mg/kg/dose (n=40), 40 mg/kg/dose (n=40), or 80 mg/kg/dose (n=40). Control mice (n=40) were treated with an i.p. injection of the Compound 003-free vehicle solution. All mice remained healthy throughout the 7-day or 30-day observation period with no evidence of morbidity. There was no significant weight loss in any of the Compound 003 treatment groups. No toxic lesions were found in any of the organs of Compound 003-treated mice. Kidney tubular degeneration was noted among ˜50% of drug-treated mice at 40 and 80 mg/kg following the 7 day period. However, insignificant amounts of tubular dilation (1/20, 1/20, 2/20 for the 80 mg/kg, 40 mg/kg and 20 mg/kg groups respectively) were noted after the 30 day observation period. Thus, the LD10 or LD50 were not reached at the 80 mg/kg/day dose level. Based on the results of the histologic evaluation of the test animals, it is concluded that Compound 003 is nontoxic in CD-1 mice at doses up to and including 80mg/kg under the conditions of this study.

TABLE 1
Acute Effects of Intraperitoneally Administered Bolus Doses of Compound 003 in CD Mice on Day 7.

| | Day 7 | | | | |
|---|---|---|---|---|---|
| | | Compound 003 Dose Level (mg/kg) | | | |
| | Vehicle | 10 | 20 | 40 | 80 |
| | (n = 20) | (n = 20) | (n = 20) | (n = 20) | (n = 20) |
| Morbidity | 0/20 | 0/20 | 0/20 | 0/20 | 0/20 |
| Toxic Organ Lesions | 0/20 | 0/20 | 0/20 | 0/20 | 0/20 |
| Weight Loss | 9/20 | 7/20 | 10/20 | 11/20 | 9/20 |
| Weight Gain | 11/20 | 13/20 | 10/20 | 9/20 | 11/20 |
| Mean Weight Change | 0.18 ± 0.25 | 0.16 ± 0.15 | −0.04 ± 0.16 | −0.06 ± 0.21 | 0.09 ± 0.20 |
| (g ± SEM [%]) | (0.7 ± 1.0%) | (0.6 ± 0.6%) | (−0.1 ± 0.7%) | (−0.2 ± 0.9%) | (0.4 ± 0.8%) |
| Bone Marrow Function | | | | | |
| WBC (×10⁹/L) | 9.2 ± 0.8 | 7.3 ± 0.6 | 8.7 ± 0.8 | 7.4 ± 0.7 | 9.5 ± 0.9 |
| ANC (×10⁹/L) | 1.3 ± 0.0 | 1.2 ± 0.0 | 1.6 ± 0.0 | 1.7 ± 0.0 | 2.4 ± 0.0 |
| ALC (×10⁹/L) | 7.5 ± 0.0 | 5.5 ± 0.0 | 6.9 ± 0.0 | 5.5 ± 0.0 | 6.7 ± 0.0 |
| RBC (×10¹²/L) | 9.2 ± 0.2 | 9.4 ± 0.2 | 9.0 ± 0.3 | 9.8 ± 0.3 | 9.3 ± 0.2 |
| Renal Function/Metabolism | | | | | |
| BUN (mg/dL) | 15.4 ± 0.9 | 24.1 ± 0.8 | 23.7 ± 0.7 | 27.6 ± 0.7 | 27.7 ± 0.9 |
| Creatinine (mg/dL) | 0.8 ± 0.4 | 0.3 ± 0.0 | 0.4 ± 0.0 | 0.5 ± 0.1 | 0.3 ± 0.0 |
| Albumin (g/dL) | 1.4 ± 0.0 | 1.5 ± 0.0 | 1.4 ± 0.0 | 1.5 ± 0.0 | 1.5 ± 0.1 |
| Total Protein (g/dL) | 4.1 ± 0.1 | 4.1 ± 0.1 | 4.2 ± 0.1 | 4.5 ± 0.1 | 4.9 ± 0.2 |
| Triglycerides (mg/dL) | 150.4 ± 29.4 | 158.3 ± 18.2 | 144.3 ± 17.5 | 134.7 ± 21.6 | 197.6 ± 34.7 |
| Calcium (mg/dL) | 7.5 ± 0.6 | 8.9 ± 0.2 | 8.9 ± 0.1 | 8.8 ± 0.2 | 9.1 ± 0.2 |
| Phosphate (mg/dL) | 9.1 ± 0.7 | 6.2 ± 0.2 | 6.6 ± 0.2 | 7.0 ± 0.2 | 6.0 ± 0.2 |
| Liver Function | | | | | |
| AST (IU/L) | 73.1 ± 12.0 | 137.1 ± 23.1 | 100.0 ± 6.7 | 134.3 ± 23.2 | 111.4 ± 7.8 |
| LDL (IU/L) | 769.9 ± 101.7 | 947.8 ± 113.5 | 1071.7 ± 112.2 | 1270.0 ± 89.0 | 1033.5 ± 73.7 |
| Total Bilirubin (mg/dL) | 0.5 ± 0.1 | 0.7 ± 0.1 | 0.5 ± 0.1 | 0.7 ± 0.2 | 0.8 ± 0.2 |
| Ammonia (μmol/L) | 532.5 ± 194.1 | 174.8 ± 11.8 | 201.9 ± 15.0 | 366.8 ± 23.7 | 293.1 ± 23.4 |
| Pancreas Function | | | | | |
| Amylase (U/L) | 1462.2 ± 50.7 | 1728.6 ± 248.5 | 1810.3 ± 268.8 | 1597.8 ± 100.0 | 1424.6 ± 37.1 |

TABLE 2
Acute Effects of Intraperitoneally Administered Bolus Doses of Compound 003
on Health, Blood Counts, and Blood Chemistry Profiles in CD-1 Mice on Day 30

| | Day 30 | | | | |
|---|---|---|---|---|---|
| | | Compound 003 Dose Level (mg/kg) | | | |
| | Vehicle | 10 | 20 | 40 | 80 |
| | (n = 20) | (n = 20) | (n = 20) | (n = 20) | (n = 20) |
| Morbidity | 0/20 | 0/20 | 0/20 | 0/20 | 0/20 |
| Toxic Organ Lesions | 0/20 | 0/20 | 0/20 | 0/20 | 0/20 |
| Weight Loss | 2/20 | 2/20 | 0/20 | 2/20 | 0/20 |
| Weight Gain | 18/20 | 18/20 | 20/20 | 18/20 | 20/20 |
| Mean Weight Change | 2.18 ± 0.41 | 2.61 ± 0.45 | 3.01 ± 0.28 | 2.34 ± 0.35 | 3.24 ± 0.29 |
| (g ± SEM [%]) | (9.1 ± 1.7%) | (10.4 ± 1.8%) | (12.4 ± 1.1%) | (9.6 ± 1.4%) | (13.4 ± 1.2%) |
| Bone Marrow Function | | | | | |
| WBC (×10⁹/L) | 9.3 ± 0.7 | 9.9 ± 1.0 | 9.3 ± 0.8 | 6.2 ± 0.7 | 5.3 ± 0.5 |
| ANC (×10⁹/L) | 1.3 ± 0.0 | 2.1 ± 0.0 | 2.0 ± 0.0 | 1.4 ± 0.0 | 1.6 ± 0.0 |
| ALC (×10⁹/L) | 7.7 ± 0.0 | 7.5 ± 0.0 | 6.8 ± 0.0 | 4.7 ± 0.0 | 3.4 ± 0.0 |
| RBC (×10¹²/L) | 8.8 ± 0.2 | 9.1 ± 0.1 | 9.1 ± 0.2 | 9.1 ± 0.1 | 9.4 ± 0.2 |
| Renal Function/Metabolism | | | | | |
| BUN (mg/dL) | 21.8 ± 0.8 | 21.6 ± 0.7 | 20.4 ± 0.6 | 20.5 ± 0.8 | 26.1 ± 1.3 |
| Creatinine (mg/dL) | 0.4 ± 0.0 | 0.3 ± 0.0 | 0.3 ± 0.0 | 0.3 ± 0.0 | 0.3 ± 0.0 |
| Albumin (g/dL) | 1.2 ± 0.0 | 1.2 ± 0.0 | 1.1 ± 0.0 | 1.2 ± 0.0 | 1.2 ± 0.0 |
| Total Protein (g/dL) | 4.0 ± 0.1 | 4.0 ± 0.1 | 3.8 ± 0.1 | 3.9 ± 0.1 | 4.1 ± 0.1 |
| Triglycerides (mg/dL) | 89.1 ± 18.1 | 88.1 ± 10.3 | 84.6 ± 7.2 | 74.9 ± 5.0 | 85.6 ± 9.5 |
| Calcium (mg/dL) | 8.3 ± 0.3 | 8.2 ± 0.2 | 8.1 ± 0.1 | 7.2 ± 0.2 | 7.7 ± 0.2 |
| Phosphate (mg/dL) | 6.2 ± 0.4 | 5.7 ± 0.2 | 6.2 ± 0.1 | 5.9 ± 0.3 | 5.8 ± 0.4 |
| Liver Function | | | | | |
| AST (IU/L) | 171.8 ± 43.9 | 114.0 ± 12.4 | 107.8 ± 13.2 | 198.5 ± 51.8 | 154.2 ± 33.8 |
| LDL (IU/L) | 723.6 ± 104.9 | 639.9 ± 55.7 | 525.0 ± 61.0 | 446.4 ± 78.4 | 383.8 ± 34.7 |
| Total Bilirubin (mg/dL) | 0.4 ± 0.1 | 0.4 ± 0.0 | 0.5 ± 0.0 | 0.5 ± 0.1 | 0.6 ± 0.0 |
| Ammonia (μmol/L) | 248.6 ± 50.0 | 205.3 ± 15.6 | 150.5 ± 10.3 | 139.2 ± 8.3 | 178.4 ± 17.9 |
| Pancreas Function | | | | | |
| Amylase (U/L) | 1214.7 ± 72.9 | 1993.5 ± 415.9 | 1587.6 ± 261.5 | 2153.7 ± 411.4 | 1397.1 ± 339.3 |

As previously mentioned, groups of 20 mice per treatment were electively sacrificed on day 7 or day 30. Blood tests performed on day 7 did not suggest any significant systemic toxicity (Table 1). Blood tests performed on day 7 & 30 (Table 1 & 2 respectively) also showed that Compound 003 did not cause (a) pathologic elevations of BUN and creatinine or electrolyte disturbances suggestive of renal toxicity, or (b) pathologic elevations of AST, LDH, or bilirubin suggestive of hepatotoxicity (Table 2). In particular, even at the highest dose level of 80 mg/kg/day and 30 consecutive days of treatment (Cumulative total dose=2.4 g/kg), there was no evidence of hematologic toxicity renal toxicity, or hepatotoxicity (Table 2). However, Compound 003 causes leukopenia in a dose-dependent fashion due to a selective depletion of lymphocytes, as reflected by a significant and dose-dependent decrease of the absolute lymphocyte count (ALC) (Table 2). The average day 30 ALC values were 7.7×10⁹/L for the vehicle-treated control group, 7.5×10⁹/L for the 10 mg/kg/day group, 6.8×10⁹/L for the 20 mg/kg/day group, 4.7×10⁹/L for the 40 mg/kg/day group, and 3.4×10⁹/L for the 80 mg/kg/day group. Thus, Compound 003 exhibits selective lymphotoxicity in CD-1 mice.

1.3.3 Toxicity of Compound 003 in Lewis Rats

Groups of 10 rats were treated with Compound 003 at 20 mg/kg/day, 40 mg/kg/day, or 80 mg/kg/day dose level for 30 consecutive days (cumulative dose: 600 mg/kg-2.4 g/kg). Results are shown below in Table 3. Two untreated rats and 10 vehicle (10% DMSO)-treated rats were included as controls. There were no immediate adverse events following any of the Compound 003 injections. All rats remained healthy and gained weight throughout the observation period with no evidence of morbidity. Blood tests done on day 30 (Table 3) did not suggest any significant systemic toxicity. In particular, even at the highest cumulative dose levels of 2.5 g/kg, Compound 003 did not cause (a) anemia, leukopenia, neutropenia, or lymphocytopenia suggestive of hematologic toxicity, (b) pathologic elevations of BUN or creatinine or electrolyte disturbances suggestive of renal toxicity, (c) pathologic elevations of AST, ALT, Alk.Ptase, or bilirubin suggestive of hepatotoxicity, or (d) pathologic elevation of amylase suggestive of pancreas toxicity. Postmortem examinations did not reveal any gross pathological findings.

TABLE 3
Cumulative Effects of Intraperitoneally Administered Bolus Doses of Compound
003 on Health, Blood Counts, and Blood Chemistry Profiles in Lewis Rats

| | | | Compound 003 Dose Level | | |
|---|---|---|---|---|---|
| | No Treatment | Vehicle | 20 mg/kg | 40 mg/kg | 80. mg/kg |
| | (n = 2) | (n = 10) | (n = 10) | (n = 10) | (n = 10) |
| Morbidity | 0/2 | 0/10 | 0/10 | 0/10 | 0/10 |
| Toxic Organ Lesions | 0/2 | 0/10 | 0/10 | 0/10 | 0/10 |
| Weight Loss | 0/2 | 0/10 | 0/10 | 0/10 | 1/10 |
| Weight Gain | 2/2 | 10/10 | 10/10 | 10/10 | 9/10 |
| Mean Weight Change (g ± SEM [%]) | 12.95 ± 1.75 | 9.36 ± 1.29 | 9.02 ± 1.39 | 9.66 ± 1.36 | 7.37 ± 1.70 |
| | (3.78 ± 0.37%) | (2.81 ± 0.39%) | (2.68 ± 0.41%) | (2.85 ± 0.38%) | (2.24 ± 0.51%) |
| Bone Marrow Function | | | | | |
| WBC (×10^{9/}L) | 13.1 ± 2.2 | 11.1 ± 0.5 | 11.9 ± 0.4 | 12.9 ± 0.4 | 12.2 ± 0.3 |
| ANC (×10⁹L) | 2.1 ± 0.0 | 1.9 ± 0.0 | 2.4 ± 0.0 | 1.9 ± 0.0 | 2.1 ± 0.0 |
| ALC (×10⁹L) | 11.0 ± 0.0 | 9.1 ± 0.0 | 9.6 ± 0.0 | 10.7 ± 0.0 | 10.0 ± 0.0 |
| RBC (×10⁹L) | 10.0 ± 0.1 | 9.7 ± 0.1 | 10.0 ± 0.1 | 10.1 ± 0.1 | 9.8 ± 0.1 |
| Liver Function | | | | | |
| ALT (IU/L) | 62.2 ± 6.2 | 59.5 ± 2.2 | 68.0 ± 5.5 | 64.2 ± 1.4 | 62.5 ± 1.8 |
| AST (IU/L) | 4.0 ± 0.0 | 3.3 ± 0.5 | 7.6 ± 1.6 | 6.8 ± 1.0 | 9.4 ± 0.7 |
| LDL (IU/L) | 318.0 ± 46.0 | 171.1 ± 24.3 | 284.3 ± 42.9 | 400.0 ± 77.8 | 318.7 ± 30.0 |
| Total Bilirubin (mg/dL) | 0.7 ± 0.1 | 0.5 ± 0.0 | 0.6 ± 0.1 | 0.6 ± 0.1 | 0.7 ± 0.1 |
| Ammonia (μmol/L) | 90.0 ± 5.0 | 97.3 ± 23.4 | 77.2 ± 4.1 | 91.4 ± 8.2 | 92.8 ± 6.0 |
| Pancreas Function | | | | | |
| Amylase (U/L) | 1487.1 ± 6.0 | 1638.8 ± 30.9 | 1667.1 ± 29.8 | 1586.3 ± 31.4 | 1658.3 ± 51.8 |
| Cardiac Function | | | | | |
| CPK (IU/L) | 361.3 ± 18.9 | 340.5 ± 39.0 | 546.1 ± 109.9 | 409.4 ± 55.3 | 371.0 ± 44.3 |
| Renal Function/Metabolism | | | | | |
| BUN (mg/dL) | 21.8 ± 0.1 | 29.1 ± 0.4 | 21.8 ± 0.4 | 21.8 ± 0.3 | 22.7 ± 0.7 |
| Creatinine (mg/dL) | 0.5 ± 0.0 | 0.5 ± 0.0 | 0.6 ± 0.0 | 0.5 ± 0.0 | 0.6 ± 0.0 |
| Albumin (g/dL) | 1.5 ± 0.0 | 1.6 ± 0.0 | 1.5 ± 0.0 | 1.6 ± 0.0 | 1.6 ± 0.0 |
| Total Protein (g/dL) | 5.9 ± 0.1 | 6.3 ± 0.1 | 6.3 ± 0.1 | 6.1 ± 0.1 | 6.3 ± 0.1 |
| Triglycerides (mg/dL) | 54.0 ± 20.0 | 84.7 ± 4.9 | 87.4 ± 9.9 | 103.0 ± 10.7 | 83.4 ± 11.2 |
| Calcium (mg/dL) | 10.4 ± 0.0 | 10.3 ± 0.2 | 10.9 ± 0.1 | 10.4 ± 0.1 | 10.5 ± 0.2 |
| Phosphate (mg/dL) | 6.3 ± 0.4 | 6.0 ± 0.5 | 6.2 ± 0.2 | 5.6 ± 0.1 | 5.9 ± 0.1 |

Lewis Rats were treated with a single i.p. bolus injection of Compound 003 for 30 consecutive days at the indicated dose levels. Rats were sacrificed on day 30. Laboratory results are presented as the mean ± SE values of laboratory parameters.

Histopathologic examinations did not reveal any toxic lesions were found in any of the 20 organs examined from the Compound 003-treated rats sacrificed on day 30, including liver, pancreas, spinal cord, spinal nerves, and skeletal muscle. Slight tubular degeneration, which may be observed in normal rats was noted in 50% of mice receiving 80 and 40 mg/kg of Compound 003, suggesting that intravenous injections of Compound 003 may promote tubular degeneration in the kidney. Taken together, these findings demonstrated that Compound 003 administered i.p. daily for up to 30 days is well tolerated by adult Lewis rats at daily dose levels as high as 80 mg/kg and cumulative dose levels as high as 2.4 g/kg.

1.3.4 Anti-Cancer Activity of Compound 003 in MMTVneu Transgenic Mice

Neu transgenic mice were mated to FVB non-transgenic mice, and heterozygous neu transgenic offspring were identified by PCR analysis. A total of 62 female neu transgenic mice were identified from the offspring. These transgenic mice were used to examine the effects of a nontoxic dose level of Compound 003 (50 mg/kg/day) on spontaneous breast cancer development in two independent experiments.

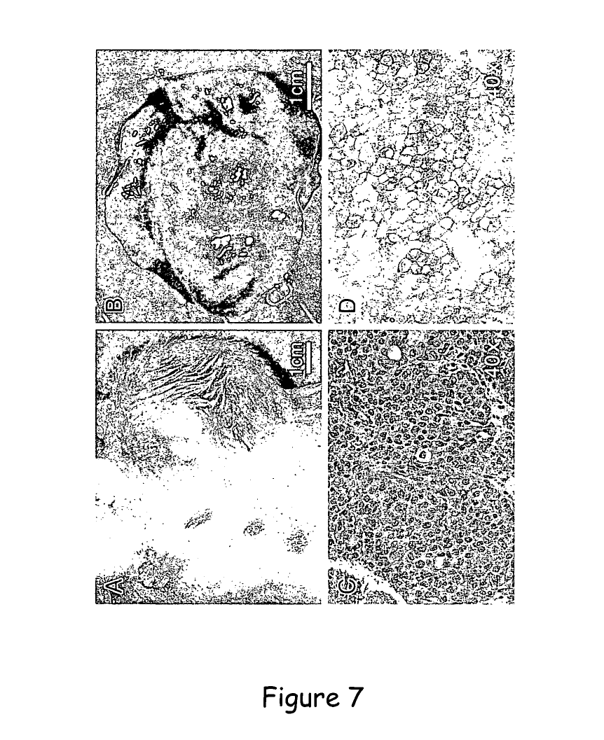

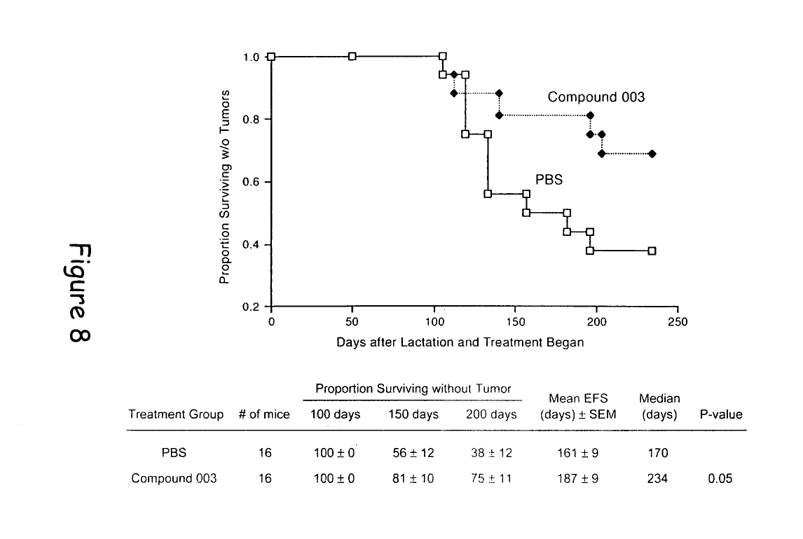

In the first experiment, 30 virgin transgenic mice were used. Treatments were initiated when female mice were 6 weeks of age. Fifteen of the 15 vehicle treated control transgenic mice developed breast cancer with a median cancer-free survival time of 224 days (Mean=253±23 days) (FIG. 7, FIG. 8A&B). At 340 days, only 20% of the mice were tumor-free and by 480 days all had developed breast cancer. The mammary gland tumors were excised, paraffin-embedded and sectioned for H+E staining. A representative tumor section from a neu transgenic mouse is shown in panel C of FIG. 8. Tumors from neu transgenic mice were typically very cellular epithelial tumors, with little fibrous stroma. Tubule and acini formation were present in <10% or 10%-75% of the tumor mass, with the majority of the tumors containing tubular structures in <10% of the tumor mass. Tumor nuclei had moderate to marked variation in size and shape. Mitotic figures-(2-6/HPF) were present in some areas. FIG. 8D illustrates the high level HER-2 expression on the surface of breast cancer cells from neu transgenic mice.

Neu transgenic mice treated, with Compound 003 developed breast cancer much later than their vehicle-treated counterparts (FIG. 7). Compound 003 prolonged the median cancer-free survival from 224 days to 455 days (P=0.0007). At 340 days, the cumulative proportion of vehicle-treated neu transgenic mice was only 20±10%, whereas at the same time 93±7% of Compound 003-treated neu transgenic mice were cancer-free. At 480 days, when all of the vehicle-treated control mice had developed breast cancer, one third of the neu transgenic mice treated with Compound 003 were still cancer-free (FIG. 7).

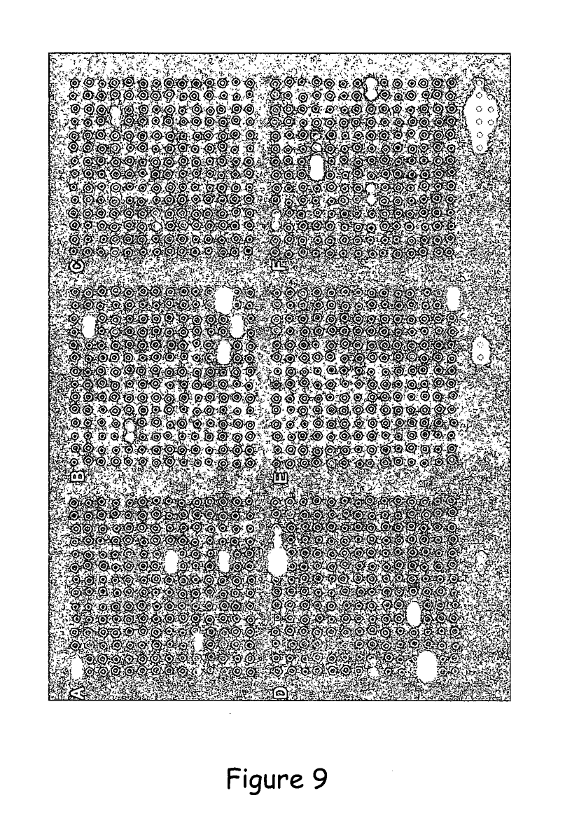

In the second experiment, in order to maintain the development of mammary gland and maximum expression of the MMTV-driven neu transgene, all female transgenic mice were kept either pregnant or lactating by continued housing with male FVB mice. Treatments commenced when female mice were 6-weeks of age. Vehicle-treated control mice (N=16) developed breast cancer faster than vehicle treated virgin transgenic mice with a median cancer-free survival time of 169.5 days (Mean=160.8±9.3 days). Compound 003 prolonged the median cancer-free survival from 169.5 days to 234 days (P=0.05). At 200 days, the cumulative proportion of vehicle-treated neu transgenic mice was only 38±12%, whereas at the same time 75±11% of Compound 003-treated neu transgenic mice were cancer-free (FIG. 9). Taken together, the results from these two experiments demonstrate that Compound 003 has marked in vivo anti-cancer activity in the MMTV/neu transgenic mouse model of breast cancer.

1.3.5 Effect of Compound 003 on Transcription Programs in Human Cancer Cells

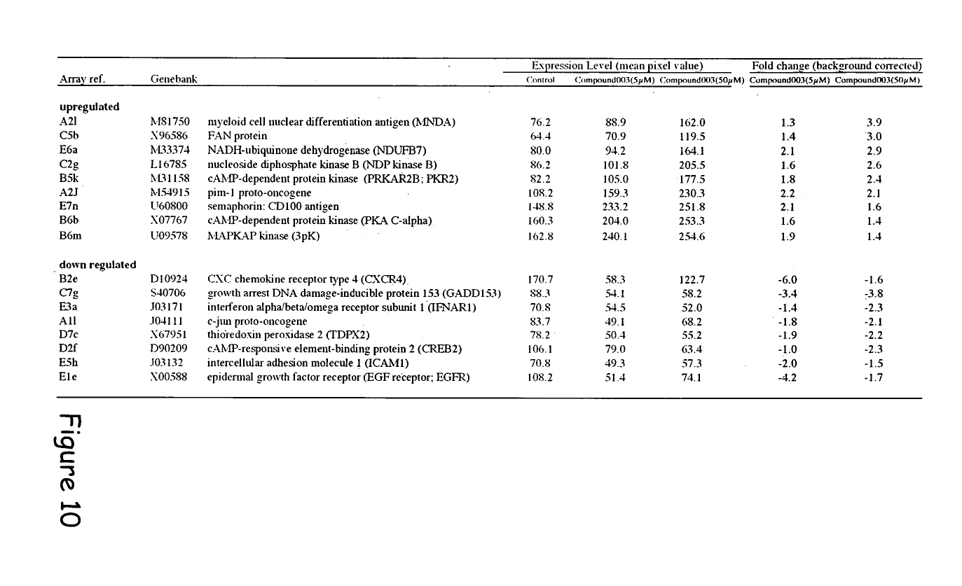

In an attempt to decipher the mechanism of action of this novel nucleoside analog we examined its effect on transcription programs in human cancer cells. Gene Profiling using the Human Atlas cDNA Expression arrays was preformed as detailed in the Materials and Methods section. Of 588 genes examined, 78 showed up-regulated expression after Compound 3 exposure. Presented below in Table 4 are the genes found to be significantly up- or down-regulated following treatment with Compound 003. Up-regulated genes were identified if the signal in the presence of Compound 003 was greater than 70 units in at least one of the samples. Notably, of these 78 genes, 16 were related to known “cell death” programs. Specifically, the gene for the TNF-R55-associated protein, FAN (Factor associated with neutral sphingomyelinase activation) (also known as CHOP) (See, for example, Werneburg, et al., 2004, Am J Physiol Gastrointest Liver Physiol. 287:6436 and Cailleret, et al., 2004, Circulation. 109:406.) which is involved in apoptotic pathways showed 3 fold increased expression following treated with Compound 003 (50 μM). We also noticed upregulated expression of 62 genes not previously linked to a particular cell death pathway. For example, at 50 μM, Compound 003 induced an almost 4 fold increase in the gene for myeloid cell nuclear differentiation antigen (MNDA) a member of the p200 (IFI-200) family of proteins that was very recently described to contain the apoptotic regulatory DAPIN (domain in apoptosis and interferon response) domain-(See, for example, Asefa, et al., 2004, Blood Cells Mol Dis. 32:155). See also, FIG. 10.

Most interesting, but somewhat conflicting is the ˜3.5 fold decreased expression of then growth arrest and DNA damage-inducible (GADD 153) gene, whose transcription factor product blocks proliferation at G1 and G2 checkpoints in response to DNA damage, particularly following endoplasmic reticulum (ER) stress ( See, for example, Terrinoni, et al., 2004, Oncogene. 23, 3721).

GADD 153 modulates apoptosis via pro- and anti-apoptotic members of the BCL2 family, through activation of caspase-3 and of c-Jun N-terminal kinase (JNK) kinase (See, for example, Ghribi, et al., 2003, J Neuropathol Exp Neurol. 62, 1144). Interestingly, the c-jun proto-oncogene, transcription factor gene product (AP-1) that signals with JNK is also downregulated ˜2 fold. These responses may be due to cellular compensation mechanisms or may suggest novel pathways involving these proteins.

Table 4 presenting genes significantly affected by Compound 003 treatment is shown in FIG. 11. Genebank accession numbers refer to National Center for Biotechnology Information (NCBI) GenBank available online at http://www.ncbi.nlm.nih.gov/. Genes that showed increases or decreases in expression relative to control were further filtered according to detection level. Up-regulated genes were identified if the signal in the presence of Compound 003 was greater than 70 units in at least one of the samples and down regulated genes showed expression levels of greater than 70 in control conditions (background approximately 50). In Table 4, background corrected fold change values relative to control are given for each drug treated condition (positive values indicate an increase in expression, negative values indicate a decrease).

1.4 Discussion

We examined the in vitro and in vivo anti-cancer activity as well as in vivo toxicity profile of Compound 003, which was identified as a candidate anti-cancer agent because of its in vitro activity in centrosomal regrowth assays. Compound 003 prevented bipolar mitotic spindle assembly and caused a G2 arrest in human cancer cells. Compound 003 was very well tolerated by both mice and rats without any toxicity at cumulative dose levels >2 g/kg. Notably, Compound 003 prolonged cancer-free survival in the MMTV/neu transgenic mouse model of HER2 positive breast cancer. The remarkable in vivo activity and safety profile of Compound 003 warrants the further development of this promising new anti-cancer agent for possible clinical use in cancer patients. Our preliminary gene profiling studies show the elements of the transcriptional program(s) activated following Compound 003 treatment. For future studies, we plan to quantify the DNA binding abilities and expression levels of the individual transcription factors that may bind to the upstream promoter regions of the co-regulated genes. As Compound 003 is a derivative of Zidovudine/AZT, these initial studies also provide further insight into the mechanism of action of Zidovudine and its derivatives.

### Example 2

2.1 Metabolism and Pharmacokinetics of Compound 003 in Cancer Cells

The metabolism and pharmacokinetics of Compound 003 in human cancer cells as well as in mice was evaluated. Dose- and time-dependent accumulation of the major intracellular metabolite ala-AZT-MP was studied in CEM T-lineage lymphoma cell line, BT-20 breast cancer cell line, T98 glioblastoma cell, line and Nalm6 B-lineage leukemia cell line following treatment with Compound 003. Dose-dependent increases in the formation of ala-AZTMP were observed in all cell lines incubated with 25-100 μM of Compound 003 for 3 hours, suggesting that no saturation of metabolism occurred with these concentrations. The intracellular accumulation of ala-AZTMP was 6-12 fold higher in BT20 breast cancer cells than the other three lines. Nalm6 leukemia cells accumulated the least amount of ala-AZTMP. Following incubation with 25 μM Compound 003, peak intracellular metabolite concentration was reached within an hour in BT20 cells, 6 hours in CEM and T98 cells, and <24 hours in Nalm6 cells. Inhibition studies were conducted in Nalm6 and BT20 cells using esterase inhibitors, bis(p-nitrophenyl)phosphate (BNPP), paraoxon, physostigmine, and phenylmethylsulfonylfluoride (PMSF).

The results demonstrated that uptake may be driven by the intracellular metabolism and that paraoxon-sensitive carboxylesterases play an important role in the conversion of Compound 003 to its major metabolite. In mice Compound 003 was rapidly converted into two metabolites, ala-AZT-MP and AZT. Maximum ala-AZT-MP concentrations were reached almost immediately (tₘₐₓ<5 minutes), while 50.4 minutes and 143.5 minutes are required to reach maximum AZT concentrations after intravenous and oral administration, respectively.

2.2.1 Chemicals

All the reagents used in this study were HPLC grade. Deionized water was prepared via a Milli-Q purification system (Medford, Mass.). Acetonitrile was purchased from Burdick & Jackson (Allied Signal Inc., Muskegon, Mich.). Methanol, acetic acid and hydrochloric acid were purchased from Fisher Chemicals (Fair Lawn, N.J.). Ammonium phosphate, phosphoric acid and AZT were purchased from Sigma (St. Louis, Mo.).

2.2.2. Synthesis and Characterization of Compound 003 and its Metabolite

Compound 3′-Azidothymidine 5′-[p-Methoxyphenyl methoxyalaninyl phosphate] (Compound 003) and ala-AZT-MP were prepared according to the literature procedure (Egron, D. et al., 1998, Bioorg Med Chem Lett. 8, 1045; McGuigan, C., et al., 1993,J Med Chem. 36, 1048; McGuigan et al.,1996, J Med Chem. 39:1748). The synthesis started with a condensation reaction of p-methoxyphenol with phosphorus oxychloride, generating phosphorodichloridate (1 in Scheme 3). Intermediate 1 in turn condensed with alanine methyl ester, furnishing phosphorochloridate 2. Condensation of 2 with AZT afforded aryl phosphoramidate derivative of AZT (Compound 003). Because of the tetrahedral configuration at the phosphorus center, the reaction product is a mixture of two diastereomers. In order to prepare COMPOUND 003-M1 (ala-AZT-MP), alkaline hydrolysis of Compound 003 afforded Compound 003-M1 (ala-AZT-MP).

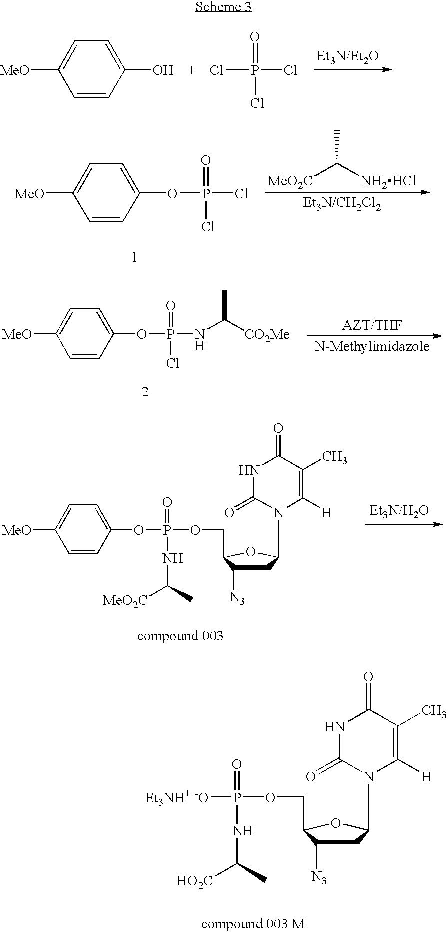

2.2.3. Experimental Data.

Analytical thin-layer chromatography (TLC) was performed on Merck precoated glass plates (silica gel 60, F₂₅₄, 250-Tm thickness), and visualized under 254-nm UV light. The preparative column chromatography was performed using EM silica gel 60, 230-400 mesh. NMR spectra were recorded on a Varian 300, using CDCl₃ with tetramethylsilane as the internal standard for ¹H (300 MHz), solvent as the internal standard for ¹³C (75 MHz), or phosphoric acid as the external standard for ³¹P (121 MHz). Mass spectra (MALDI-TOF) were recorded by a G2025A LD-TOF system (Hewlett Packard). MS samples were prepared with 1:1=matrix (30-mM α-cyano-4-hydroxycinnamic acid in 50%:30% :20%=acetonitrile:methanol:water): NMR sample in CDCl₃. HPLC data was recorded using an HP 1100 system, with a Zorbax SB-C3 column (5 Tm, 4.6×150 mm), with water (50 mM NH₄H₂PO₄/H₃PO₄, pH 2.5):acetonitrile=70:30, for COMPOUND 003 and 92:8 for ala-AZT-MP with a flow rate of 1 mL/min and detection by UV at 218 nm for COMPOUND 003 and 208 nm for ala-AZT-MP. Reactions were performed in glassware which had been oven dried (120° C./overnight), and under nitrogen atmosphere. All reaction mixtures were stirred magnetically, unless otherwise noted.

Solvents and reagents were used as purchased from Aldrich Chemical Co., unless otherwise stated. The yields quoted in this paper were isolated yields. Roth COMPOUND 003 and AZT-5′-alaninylphosphase (ala-AZT-MP) were identified by NMR, GC/MS, IR and UV spectrometry. The chemical structures were presented above in Scheme 1. The purity was further confirmed by thin layer chromatographic technique (TLC) and HPLC and was found to be over 95.0%.

3′-Azidothymidine 5′-[p-Methoxyphenyl methoxyalaninyl phosphate] (COMPOUND 003). ¹H NMR (CDCl₃). Starred peaks are split due to diastereoisomers.) 6 8.35 (s, 1H), 7.37 (t, J=1.5 Hz, 1H), 7.15-7.10 (m, 2H), 6.85-6.82 (m, 2H), 6.21 (t, J=6.6 Hz, 2/3H), 6.15 (t, J=9.0 Hz, 1/3H), 4.37-4.33 (m, 2H), 4.07-4.01 (m, 1H), 3.78* (s, 3H), 3.73 (s, 1H), 3.72 (s, 2H), 3.63 (t, J=11.1 Hz, 1H), 2.42-2.34 (m, 1H), 2.24-2.14 (m, 1H), 1.91 (d, J=1.2 Hz, 2H), 1.90 (d, J=0.9Hz, 1H), 1.39 (d, J=7.5 Hz, 1H), 1.36 (d, J=6.9 Hz, 2H); ¹³C NMR (CDCl₃) 6 173.9, 173.7, 163.6, 156.7, 150.1, 143.7, 143.6, 135.2, 120.9, 120.8, 111.3, 107.2, 85.0, 84.7, 82.3, 82.2, 82.1, 77.2, 65.7, 65.6, 65.5; 60.4, 60.3, 55.6, 52.6, 50.4, 50.2, 37.3, 21.0, 20.9, 12.5; ³¹P NMR (CDCl₃) 3.3, 3.0; MALDI-TOF MS m/z 539.0 (MH⁺), 561.1 (MNa⁺); HPLC retention time 10.8 (50%/o), 11.2 (50%) min; UV λₘₐₓ 192, 216, 268 nm.

Triethylammonium 3′-Azidothymidine 5′-[Alaninyl phosphatel (ala-AZT-MP). ¹H NMR (DMSO-d₆) δ 11.30 (bs, 1H), 7.81 (s, 1H), 6.15. (t, J=6.6 Hz, 1H), 4.50 (q, J=6.6 Hz, 1H), 3.95 (d, J=5.7 Hz, 1H), 3.84 (t, J=2.4 Hz, 2H), 3.45-3.34 (m, 2H), 2.77 (q, J 6.9 Hz, 6H), 2.44-2.24 (m, 2H), 1.78 (s, 3H), 1.11 (d, J=6.6 Hz, 3H), 1.06 (t, J=7.2 Hz, 9H); HPLC retention time 11.8 min; UV λₘₐₓ 208, 268 nm.

2.2.4 Cell Culture and Drug Treatment

Intracellular metabolism of Compound 003 was studied in CEM T-lineage lymphoma cell line, BT20 breast cancer cell line, T98 glioblastoma cell line, and Nalm-6 B-lineage leukemia cell line. CEM and Nalm6 cells were maintained in RPMI supplemented with 10% fetal bovine serum, and 1% penicillin/streptomycin. BT20 and T98 cells w.ere cultured in a medium composed of MEM, 10% fetal bovine serum, 0.1-mM of nonessential amino acids and Earles salts, and 1% penicillin/streptomycin. CEM and Nalm6 cells at a density of 10⁶ cells per ml were utilized in the drug metabolism study. BT20 and T98 cells were seeded in 6-cm petri-dishes at 4 million cells and 2 million cells per plate, respectively, the day before the experiment. Either one plate of adherent cells or 10 million non-adherent cells was utilized at each time point. Cells were incubated with various concentrations of the compounds up to 24 hrs at 37° C. After incubation, cells were washed 2 times with ice-cold PBS, and extracted by addition of 0.5-1 ml of 60% methanol. Cell lysates were kept at −20° C. overnight, after which lysates were centrifuged at 15000×g for 10 minutes to remove the cell debris. One hundred microliters of these lysates were injected directly to HPLC. For half-life study, cells at the same density as in drug metabolism study were treated with 100 μM Compound 003 for 2 hrs, after which cells were washed 2 times with ice-cold PBS, and resuspended in warm media until the time they were harvested. For uptake and efflux study, cells were seeded at 2 million cells in 2 ml of media. Cells were incubated with 50 μM Compound 003 for 1 hr, and the amount of parent compound left in the medium was then subtracted from that in the medium alone without cells containing 50 μM Compound 003, to measure the cellular uptake of Compound 003. With efflux experiments, cells 1×10⁶/mL (the same density as in the uptake study) were incubated with 100 μM Compound 003 for 1 hr, washed 2 times with ice-cold PBS, and resuspended with culture medium without drug, and the metabolite efflux from cells to the medium was measured after 6 hr of incubation.

2.2.5 Preparation of Cell Homogenate for Compound 003 Metabolism and Esterase Assay

Cells were homogenized in 10 mM Tris-HCl (pH 7.4) containing 250 mM sucrose using sonicator. Homogenates were centrifuged at 10,000×g in Sorvall RMC14 microfuge. Supernatants were utilized for COMPOUND 003 metabolism and esterase assays. For the COMPOUND 003 metabolism essay, 50 μl of cell lysates in 200 μl Tris buffer (pH 7.4) was incubated with 1 mM COMPOUND 003 for 2 hr, after which 125 μl acetone was added. After removal of the protein precipitation, 100 μl was injected into HPLC, and the data was expressed as nmol per mg cellular protein. For the esterase assay, 10 μl of cell homogenates in 190 μl Tris buffer (pH 7.4) was incubated with 1 mM para-nitrophenyl acetate for 15 minutes in 96-well plates. Reactions without cell homogenates serve as controls. This enzyme assay was linear with respect to protein and time, and the rate of formation of p-nitrophenol was monitored every minute at the wavelength of 405 nm.

2.2.6 Identification and HPLC Analysis of Alaninyl-AZT Monophosphate

The HPLC system consisted of a Hewlett Packard (HP) 1100 series equipped with a quarternary pump, an auto sampler, an electronic degasser, a diode-array detector, and a computer with a chemstation software program for data analysis (McGuigan, C., et al., (1996) Bioorg Med Chem Lett. 6:1183; Chen, C. L., et al., (1999) J Chromatogr B Biomed Sci Appl. 724:157). The samples were eluted on a 250×4.6 mm Zorbax® SB-C18 column. A solvent gradient was utilized to resolve the metabolite from the parent compound, which consisted of a mixture of methanol and 10 mM ammonium phosphate (pH 3.7). The gradient ran at a flow rate of 1 ml per minute from 5 to 35% methanol for the first 8 minutes, kept at 35% methanol for 4 minutes, and finished with a linear gradient from 35 to 100% methanol in the next 20 minutess. The retention time of ala-AZT MP was at about 12 minutes. An alternative method was used for some experiments, consisting of 5 to 18% acetonitrile in ammonium phosphate buffer over the first 8 minutes, kept at 18% from 8-12 minutes, finishing with a linear gradient from 18 to 100% in the next 20 minutes. The retention time of ala-AZT-MP using this method was about 10 minutes, that of AZT was about 13 minutes, and that of the minor metabolite AZT-MP was about 7.6 minutes. The detection wavelength was set at 278 nm.

2.2.7 Analysis of Metabolism using Esterase Inhibitors:

BT20 breast cancer cells were cultured in a medium composed of MEM, 10% FBS, 0:1 mM of nonessential amino acids and Earles salts, and 1% penicillin/streptornyclin Nalm6 cells were maintained in a medium composed of RPMI, 10% fetal bovine serum (FBS) and 1% penicillin/streptomycin. BT20 cells were seeded in 6-cm petri-dishes at 4 million cells per plate. Nalm6 cells at a density of 10⁶ cells per ml were also utilized in drug metabolism study. Either one plate of adherent cells or 10 million nonadherent cells were utilized at each experiment. Cells were incubated with DMSO or 100 μM of esterase inhibitor, paraoxon, bis(p-nitrophenyl)phosphate (BNPP), physostigmine, or phenylmethylsulfonyl fluoride (PMSF) (Sigma) for 3.0 minutes, followed by 1 hour incubation of 100 μM Compound 003. After incubation, cells were washed two times with ice-cold PBS, and extracted by addition of 60% methanol. Cell lysates were kept at −20° C. overnight, after which lysates were centrifuged at 15,000×g for 10 minutes to remove the cell debris. One hundred microliters of these lysates were injected directly to HPLC. The amount of metabolite product of the esterase reaction, ala-AZT-MP, was measured to examine the effect of inhibitors on esterase-mediated metabolism of Compound 003.

Intracellular metabolism of Compound 003 was studied in Nalm6 cells. Nalm6 cells were maintained in a medium composed of RPMI, ₁₀% fetal bovine serum, and 1% penicillinistreptomycin. Ten million cells at the density of 1 million/ml were utilized in each experiment of time and concentration-dependent studies. Cells were incubated with various concentrations of these compounds up to 24 hr at 37° C. After incubation, cells were washed 2 times with ice-cold PBS, and extracted by addition of 250 μl of 60% methanol. Cell lysates were kept at −20° C. overnight, after which lysates were centrifuged at 15000×g for 10 minutes to remove the cell debris. One hundred microliters of these lysates were injected directly to HPLC. Areas under curve (AUC) were calculated from time-dependent metabolism data by trapezoidal rule using Prism software.

2.2.8 Preparation of Subcellular Fractions

1−3×10⁸ Nalm6 and BT20 cells were washed with PBS, resuspended in homogenizing buffer containing 10 mM Tris-HCl (pH 7.4) and 250 mM sucrose. Cells were sonicated and spun at 10,000×g, and the resulting supernatant was centrifuged again at 100,000×g to isolate microsomal and cytosolic fractions. The microsomal pellet was resuspended in a buffer containing 10 mM Tris-HCl (pH 7.4), 250 mM sucrose, 1 mM EDTA, and 20% glycerol. Microsomes or cytosols of Nalm6 (80 μg. and 86 μg, respectively) and BT20 cells (59 μg and 160 μg, respectively) were preincubated with or without 1 mM paraoxon in Tris-HCl buffer (pH 7.4) in a total volume of 250 μl for 10 minutes; 1. mM COMPOUND 003 was then added to the incubation mixture, and the reactions were allowed to proceed for another 30 minutes and terminated by addition of 125 μl of acetone. Protein precipitation was then removed by centrifugation; 100 μl of the supernatant was injected directly into the HPLC.

2.2.9 Enzyme Kinetics of Compound 003 Metabolism Microsomal or cytosolic fractions of Nalm6 and BT20 cells were incubated with COMPOUND 003 at various concentrations from 10 μM to 1.2 mM in similar reaction conditions as described above, except without the inhibitor. The reactions were allowed to proceed for 40 minutes. Km and Vmax in the Michaelis-Menten model were estimated by WinNonlin program (Scientific Consulting, Inc.)

2.2.10 Isolation and Identification of the Plasma Metabolite (Compound 003-M1).

CD-1 mice were injected intravenously with COMPOUND 003 at dose of 200 mg/kg. At 10 minutes, blood was collected from the ocular venous plexus by retro-orbital venipuncture, and the plasma was obtained by centrifugation. Then 2 volume of acetonitrile was added to the pooled plasma to precipitate the plasma protein. The acetonitrile was evaporated under the nitrogen gas stream. Then extraction by ethyl estate ( 2 times) was used to remove any components which can be dissolved in ethyl estate. The aqueous layer contained Compound 003-M1 was lyophilized to dry and reconstituted in methanol. The reconstituted solution was analyzed in Hewlette Packard LC-MS. The MS conditions were set at fragmentor of 60, drying gas flow of 10 L/min, nebulizer pressure of 25 psig, drying gas temperature of 350° C. gas of 10 L/min, temperature of 350° C. Peak width and gain were set at 0.03 s and 1.0, respectively.

2.2.11 Isolation of Urinary Metabolite (Compound 003-M2). Ten CD-1 mice were placed into a Nalgene metabolic cage after being injected intravenously with 200 mg/kg Compound 003 by i.v. injection. Urine was collected at 0-24 h. The urine was centrifuged and stored at −20° C. An aliquot of the urine sample was extracted with ethyl acetate and the organic phase was transferred to a clean tube. The ethyl acetate extract was dried under nitrogen gas and the residue was used directly for NMR analysis.

25 2.2.12 NMR Analysis. ¹H-NMR spectra was measured on a Model Varian XL-400 NMR spectrometer operating at 399.9 MHz and 100.6 MHz, respectively, and employing a deuterium field-lock frequency in the normal manner. The samples were dissolved in DCCl₃.

2.2.13 HPLC Determination of Compound 003 and its Metabolites in Plasma

Each plasma sample (200 μL) was mixed 1:4 with acetone (800 μL) and vortexed for at least 30 seconds. Following centrifugation, the supernatant was transferred into a clean tube and dried under nitrogen. A 50 μL solution of 50% methanol in 200 mM HCl was used to reconstitute the extraction residue, and 35 μL was subjected to analytical HPLC. The analytical column used was a 250×4 mm Lichrospher 100, RP-18 (5 μm) and the guard column was a 4×4 mm Lichrospher 100, RP-18 (5 μm). The mobile phase was degassed automatically by the electronic degasser system. The column was equilibrated and eluted under gradient conditions utilizing a flow rate of 1.0 ml/min at ambient temperature. The linear gradient mobile phase was: 0 minute, 100% 10 mM ammonium phosphate buffer (pH 3.7); 20 minutes, 20% acetonitrile, 80% ammonium phosphate buffer (pH 3.7); 20.1-50 minutes, 28% acetonitrile, 72% water (0.1% HAC). The wavelength of detection was set at 270 nm. Peak width, response time and slit werep, set at >0.03 minutes, 0.5 s and 8 nm, respectively.

A calibration curve was generated to confirm the linear relationship between the peak area and the concentration of Compound 003 and its two metabolites in the test samples. Compound 003 and its two metabolites, ala-AZT-MP and AZT were added to plasma to yield final concentrations of 0.25, 0.5, 1.25, 2.5, 5.0, 12.5,25, 50 and 100 μM following precipitation of plasma by acetone. Subsequently, the plasma samples with known amounts of drugs were extracted as described above, and the standard curves were generated by plotting the peak area of COMPOUND 003 and its metabolites, against the drug concentrations tested. Unweighted linear regression analysis of the standard curve was performed by using the CA-Cricket Graph III computer program, Version 1.1 (Computer Association, Inc., Islandia, N.Y.). The linearity-was confirmed by testing for departure from linearity using the Instat Program V3.0 (GraphPad Software, San Diego, Calif.).

2.2.14 Stability of Compound 003 in Plasma, Gastric Fluids and Intestinal Fluids

Plasma samples, simulated gastric fluid and intestinal fluids (United State Pharmacopeia XXII methods) were separately spiked with COMPOUND 003 to yield final concentrations of 500 μM. Spiked samples were incubated at 37° C. At a predetermined time (5, 10, 15, 30 60 and 120 minutes), an aliquot (200 μl) of the spiked plasma sample was extracted as described above.

2.2.15 Evaluation of Pharmacokinetics and of Compound 003 in Mice

Female CD-1 mice (6-8 weeks old) (Charles River, Wilmington, Mass.) were housed in a USDA-accredited animal care facility under standard environmental conditions (12-h light/12-h dark photoperiod, 22±1° C., 60±10% relative humidity). All rodents were housed in microisolator cages (Lab Products, Inc., N.J.) containing autoclaved bedding. Mice were allowed free access to autoclaved pellet food and tap water throughout the study. All animal studies are approved by the Parker Hughes Institute Animal Care and Use Committee, and all animal care procedures conformed to the principles outlined in the Guide for the Care and Use of Laboratory Animals (National Research Council;. National Academy Press, Washington D.C. 1996).

Compound 003 (200 mg/kg) dissolved in DMSO was administered intravenously via tail vein to non-fasted mice or orally via gavage to 12-h fasted mice. Four to six mice per time-point were used for pharmacokinetic studies. Blood samples (˜500 μL) were obtained from the ocular venous plexus by retro-orbital venipuncture at 0, 5, 10, 15, 30, 45 minutes, and 1 h, 1.5 h, 2 h, 4 h, 6 h and 8 h after oral administration. All collected blood-samples were heparinized and centrifuged at 7000×g for 5 minutes to separate the plasma fraction from the whole blood. The plasma samples were then processed immediately using the extraction procedure described above.

2.2.16 Pharmacokinetic Analysis

Pharmacokinetic modeling and parameter calculations were carried out using the WinNonlin Professional Version 3.0 (Pharsight, Inc., Mountain, Calif.) pharmacokinetics software (Chen, C. L., et al., (1999) Pharm Res. 16:117; Chen, C. L., et al., (1999) Pharm Res. 16:1003; Uckun, F. M., et al., (1999) Clin Cancer Res. 5:2954). An appropriate model was chosen on the basis of the lowest sum of weighted squared residuals, the lowest Schwartz Criterion (SC), the lowest Akaike's Information Criterion (AIC) value, the lowest standard errors of the fitted parameters, and the dispersion of the residuals. The elimination half-life was estimated by linear regression analysis of the terminal phase of the plasma concentration-time profile. The systemic clearance (CL) was determined by dividing the dose by the AUC.

2.3 Results

2.3.1 Dose- and Time-Dependent Accumulation of Ala-AZT-MP in Human Cancer Cells

Lysates from cells incubated with Compound 003 were analyzed by the HPLC method described above. Minimal amounts of the parent drug were detected in these lysates, whereas a major metabolite peak appeared at about 12 minutes. This peak co-eluted with synthesized alanine-AZT-monophosphate (ala-AZT-MP); both have identical UV spectra and mass spectra. Based on this result, we concluded that Compound 003 is a prodrug, which is rapidly metabolized to ala-AZT-MP intracellularly. The rate and extent of metabolism of the major intracellular metabolite was then assessed by analysis of ala-AZT-MP. As shown in FIG. 12A, dose-dependent proportional increase in the formation of ala-AZT-MP was observed in all cell lines incubated with 25, 50, and 100 μM at 3 hr. The intracellular accumulation of ala-AZT-MP was 6-12 fold higher in BT20 cells than in the other three cell lines. Comparable levels of the metabolite were seen in CEM and T98 cells, Nalm6 cells accumulated the least amount of ala-AZTMP. Time-dependent Compound 003 metabolism was studied up to 24 hr at 25 and 50 μM. Different patterns of time-dependent accumulation of ala-AZT-MP were observed (FIG. 12B). While a fast-rising and then decreasing trend over time in intracellular concentrations of ala-AZT-MP was seen in BT20 cells, the accumulation of the metabolite was gradually increasing in Nalm6 cells. The pattern of metabolite accumulation was similar in T98 and CEM cells, except at 50 μM for 24 hr, where, increasing concentration of the metabolite was seen in CEM cells but not T98 cells. Peak intracellular metabolite concentration in BT20 cells incubated with 25 μM

As shown in Table 5, BT20 had 9-fold higher uptake of Compound 003 than T98 cells, whereas Nalm6 cells had similar uptake as CEM cells. The half-life of the intracellular metabolite in BT20 cells was similar to that of T98 cells; similar half-lives were also seen in Nalm6 and CEM cells. In addition, efflux of ala-AZT-MP was only detectable from BT20 cells, which may explain the rapid decline of ala-AZT-MP in this cell line. These results are consistent with those obtained in dose- and time-dependent metabolism experiments, where faster formation of the intracellular metabolite was observed in BT20 cells as compared to T98 cells. The time-dependent decline of ala-AZT-MP in BT20, T98 and CEM cells is most likely related to gradual depletion of the parent compound in the media and efflux of the metabolite from the cell. However, since this data alone cannot fully explain the faster rates of metabolite formation in CEM cells as compared to Nalm6 cells, the following experiments were conducted. Cell homogenates were utilized to study whether intracellular factors may be related to the rates of Compound 003 metabolism in these cell lines. Results are shown in Table 6.

TABLE 6
Estimated Pharmacokinetic Parameter Values
Following Treatment With COMPOUND 003

| | AUC | Cₘₐₓ | t_{1/2} | tₘₐₓ |
|---|---|---|---|---|
| Measured | (μM · h) | (μM) | (h) | (min) |
| Following i.v. | | | | |
| Ala-d4T-MP | 190.9 ± 17.4 | 1879.8 ± 214.4 | 33.2 ± 2.8 | 1.2 ± 0.1 |
| AZT | 152.0 ± 31.0 | 64.3 ± 6.9 | 35.0 ± 3.2 | 50.4 ± 4.6 |
| Following p.o. | | | | |
| Ala-d4T-MP | 20.7 ± 8.5 | 9.8 ± 1.9 | 94.5 ± 32.0 | 4.5 ± 0.4 |
| AZT | 386.0 ± 123.7 | 46.9 ± 5.4 | 148.7 ± 8.3 | 143.5 ± 41.0 |

Pharmacokinetic parameters in CD-1 mice (n=5-6 mice per time point) are presented as the mean ±S.E. Abbreviations: t_{1/2} is terminal elimination half-life; tₘₐₓ is the time required to reach the maximum plasma drug concentration following injection.

Homogenates from BT20 cells incubated with Compound 003 displayed a 9-fold higher level of metabolite formation as compared to T98 cell homogenates. CEM cell homogenates had 2-fold higher accumulation of metabolite than Nalm6 homogenates (Table 6). The consistency between the metabolism data derived from intact cells and cell homogenates indicates that accumulation of ala-AZT-MP is not directly related to uptake per se, but rather correlates with the extent of intracellular metabolism. In order to determine if esterases could be involved in the metabolism of Compound 003, esterase activity was measured using p-nitrophenylacetate (PNPA) as a substrate (Morgan, E., et al., (1994) Arch Biochem Biophys. 315:495; McCracken, N. et al., (1993) Biochem Pharmacol. 46:1125). As shown in Table 5, BT-20 had 3-fold higher pNPA hydrolytic activity than T98 cells, whereas similar esterase activities were observed in CEM and Nalm6 cells. These results support the hypothesis that esterases may be involved in the metabolism of Compound 003.

To assess whether additional down-stream pathways contribute to the overall metabolism of Compound 003, we studied intracellular production of other minor metabolites. As shown in Table 5, ala-AZT-MP preferentially converted to AZT-MP in BT20 cells, but to AZT in T98 cells. In addition, ala-AZTMP was converted to AZT first then to AZTmp in Nalm6 cells, but only AZT was detectable in CEM cells. Therefore, the metabolic rate from AZT to AZT-MP appeared to be faster in Nalm6 than in CEM cells.

2.3.2 Involvement of Paraoxon-sensitive Esterases in Metabolism of Compound 003

To test the hypothesis that carboxylesterases are responsible for the metabolism of Compound 003, four esterase inhibitors were utilized in the present study. BNPP is an inhibitor of carboxylesterase isoenzymes (Gaustad, R. et al. (1991) Biochem Pharmacol. 42:1335), paraoxon, the active metabolite of parathion, a potent inhibitor of carboxylesterase and cholinesterase (Kaliste-Korhonen, E., et al., (1996) Hum Exp Toxicol. 15:972; Ehrich, M., et al., J Toxicol Environ Health A. (1998) 53:385), physostigmine a cholinesterase inhibitor (Williams, F. M., Clin Pharmacokinet. (1985) 10:392), and PMSF an inhibitor of microsomal carboxylesterases (Morgan, E. et al., (1994) supra). The profiles of esterase inhibition by these inhibitors are carried out in two human cell lines, BT20 and Nalm6 cells. As shown in FIG. 13, the metabolism of Compound 003 to ala-AZT-MP was inhibited significantly (60-800%) by paraoxon in BT20 cells, but not by BNPP or PMSF. These results suggest that carboxylesterases but not cholinesterases mediate the metabolism of Compound 003 in BT20 cells. Similar results were observed in Nalm6 cells with paraoxon being the -most potent inhibitor in the metabolism of Compound 003.

Since carboxylesterases are present in both microsomal and cytosolic fractions (McCracken, N. et al. (1993) supra), these two subcellular fractions were utilized to further characterize the carboxylesterases responsible for the metabolism of Compound 003. Microsomes and cytosols of BT20 and Nalm6 cells in appropriate subcellular proportions were incubated with Compound 003. Our results showed that ala-AZT-MP formation was detected following Compound 003, incubation with either cytosols or microsomes from BT20 cells. To determine the relative contribution of microsomes and cytosol to the overall metabolism of Compound 003 in these cells, enzyme kinetics were conducted in these two subcellular fractions. In BT20 cells, a Kin of 460 μM vs. 1751 μM and Vmax of 0.7 vs. 5.9 nmol/mg/min were obtained for microsomal and cytosolic fractions, respectively. In Nalm6 cells, a Km of 163 μM vs. 765 μM and Vmax of 82.3 vs. 541 pmol/mg/min were obtained for microsomal and cytosolic fractions, respectively.

2.3.3 Identification of In Vivo Metabolites of Compound 003

The Compound 003 metabolite (Compound 003-Mich.) extracted from plasma following i.v. injection of Compound 003 was analyzed by LC-MS. The MS spectrum shown that molecular weight for Compound 003-M1 was 419 (FIG. 14A), which was the same spectrum as the synthetic authentic compound (3′-azido-3′-deoxythymidine 5′-alaninylphosphate) under the same LC-MS conditions (FIG. 14B). Therefore, Compound 003-M1 was identified as 3′-azido-3′-deoxythymidine-5′-alaninyl phosphate (ala-AZT-MP).

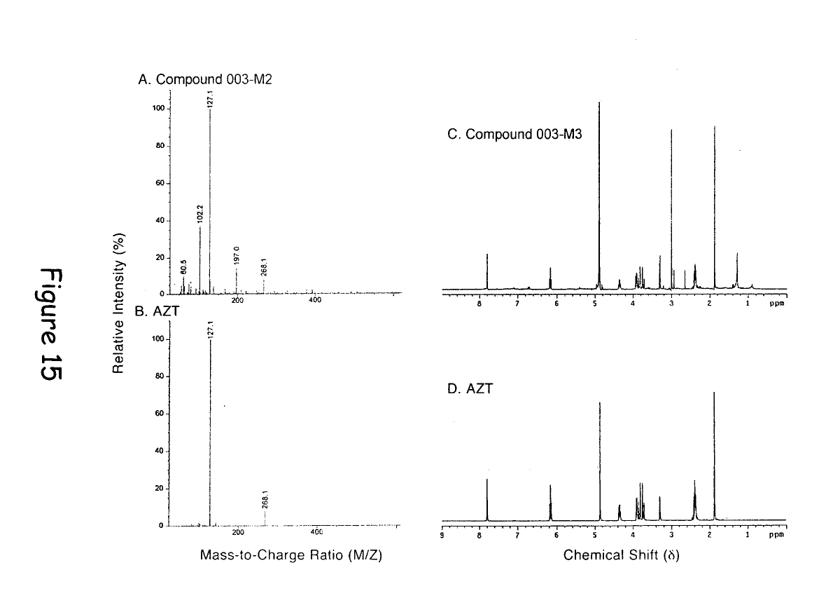

The second metabolite (Compound 003-M2) was extracted directly with ethyl acetate and MS spectrum was identical as that of pure AZT (FIG. 15A vs 15B). This is also confirmed by the NMR spectrum (FIG. 15C vs 15D). Therefore, Compound 003-M2 was identified as AZT.

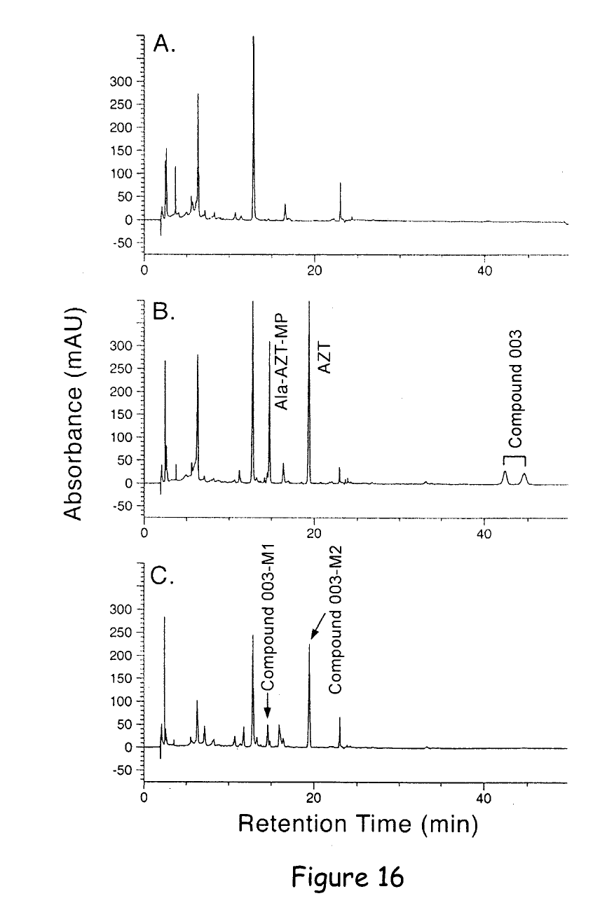

Using extraction described in the Materials and Methods section, the extraction recoveries (mean±SD) of ala-AZT-MP, AZT and Compound 003 from plasma were 64.6±2.0%, 85.5±1.2% and 78.9±2.1%, respectively. We established standard HPLC conditions for simultaneous separation of Compound 003 and its metabolites, ala-AZT-MP and AZT, in plasma. Using the chromatographic separation conditions described in the Materials and Methods section, the retention times (R_{T}) (±SD) measured for Compound 003 (two diasteromers) and its metabolites in spiked samples were 42.1±0.8 minutes (Compound 003-A; n=12), 44.3±0.9 minutes (Compound 003-B; n=12), 14.6±0.1 minutes (ala-AZT-MP; n=27) and 19.3±0.2 minutes (AZT; n=27), respectively. At these retention times, no significant interference peaks were observed in the blank plasma samples (FIGS. 16B vs 16A).

The hydrochloric acid component of the reconstituted solutions plays a key role in the chromatography of Compound 003 and its metabolites, as the acid protonates ala-AZT-MP and no peak appears in the chromatogram for this metabolite if there is no hydrocholoric acid in the reconstituted solution. The acidic solution renders ala-AZT-MP less stable, therefore, all of the extracted samples were analyzed immediately after reconstitution.

The lowest limit of detection was 0.5 μM at S/N ˜4. Good linearity (r>0.995) was observed between concentrations ranging from 0.5 μM to 12.5 μM and from 12.5 μM to 100 μM in 200 μl plasma (standard curves and linear equations are not shown). The linearity was statistically confirmed using the Instat Program V3.0.

2.3.4 Stability of Compound 003 in Biological Fluids

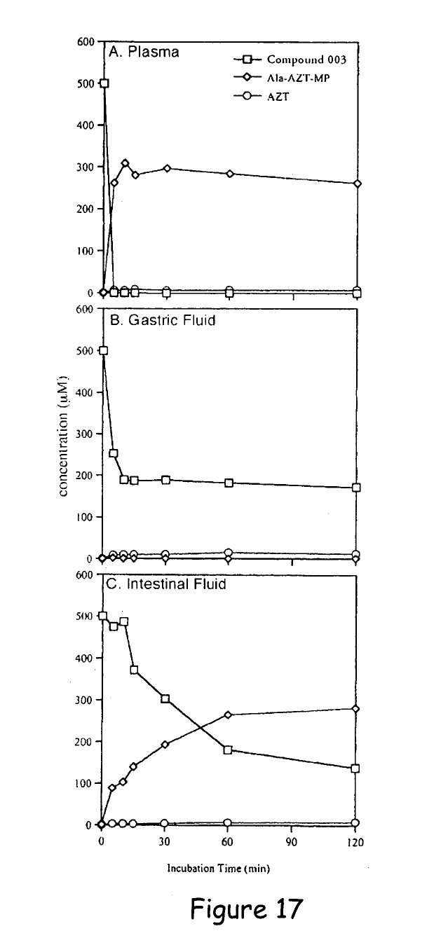

The results shown in FIG. 17A indicate that Compound 003 is unstable in plasma. Following incubation with plasma, >99% of Compound 003 decomposes within 5 minutes. Hence, immediate extraction of the samples is required after collection in order to accurately measure the Compound 003 levels. In plasma,. Compound 003 decomposed to mainly form ala-AZT-MP.

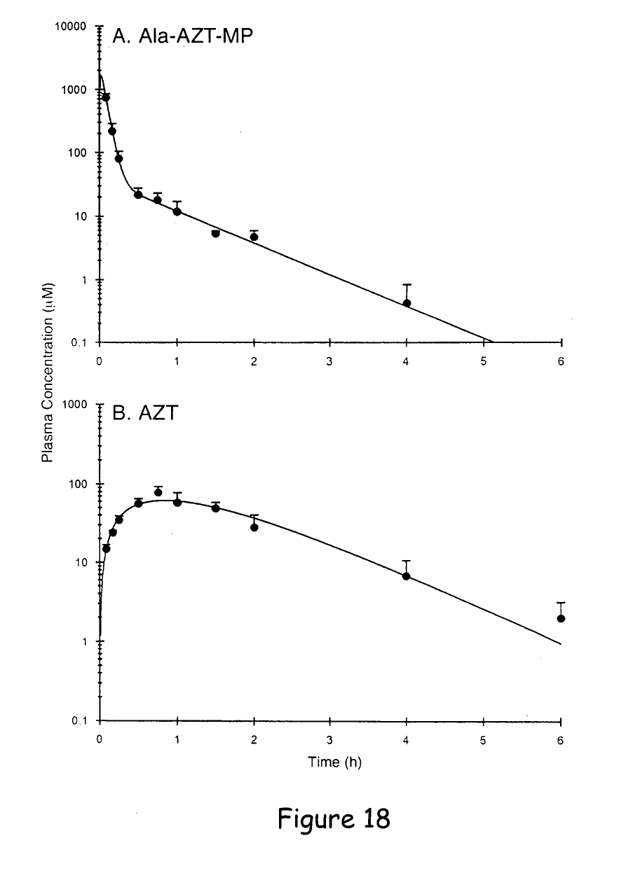

The results of the stability studies also revealed that Compound 003 is also unstable in simulated gastric fluid and intestinal fluid (FIGS. 18B & 18C). In gastric fluid, Compound 003 quickly decomposed to yield a intermediate product at retention time of 23 minutes which was not ala-d4T-MP or AZT. The chemical identity for this intermediate product is unknown. In intestinal fluid, Compound 003 decomposed to form ala-AZT-MP.

2.3.5 Pharmacokinetic Profiles of Compound 003 in Mice Following Intravenous Administration

Following i.v. injection of 200 mg/kg, Compound 003 was immediately transformed to Ala-AZT-MP and AZT. The plasma Ala-AZT-MP concentration-time curve is presented in 17A, and can be described as a two-compartment model. The pharmacokinetic parameter values are presented in Table 6. Following i.v. bolus injection of 200 mg/kg of Compound 003 to CD-1 mice, Ala-AZT-MP showed moderate elimination (t_{1/2} of 33.2 minutes). The AUC and predicted Cₘₐₓ were found to be 190.9 μM·h and 1879.8 μM, respectively.

Following intravenous administration of Compound 003 at 200 mg/kg, the plasma AZT concentration-time curve can be best described by a one-compartmental model (FIG. 18B). The pharmacokinetic parameter values were presented in Table 6. The predicted Cₘₐₓ and AUC of AZT in mice were 64.3 μM and 152.0 μM*h. Maximum plasma AZT concentration following Compound 003 administration was reached at 50.4 minutes. The elimination half-life of AZT was 35.0 minutes.

2.3.6 Pharmacokinetic Profile of Compound 003 Following Oral Administration

Both metabolites (ala-AZT-MP and AZT) were detected in the plasma of animals following oral administration of 200 mg/kg DDE 3 , but the concentration of parent Compound 003 was below the detection limit (0.5 μM). A one-compartment pharmacokinetic model was used to describe the time-dependent concentration changes for ala-AZT-MP and AZT (FIGS. 19A and 19B). The tₘₐₓ values were 4.5 minutes for ala-AZT-MP and 143.5 minutes for AZT. The estimated values for the pharmacokinetic parameters are presented in Table 6. The maximum concentrations (Cₘₐₓ) for ala-AZT-MP and AZT are 9.8 μM and 46.9 μM, respectively. The elimination half-lives were 94.5 minutes and 148.7 minutes for ala-AZT-MP and AZT, respectively.

2.4 Discussion

The metabolism of the AZT-phosphoramidate Compound 003 was studied in four tumor cell lines. No saturation of metabolism was observed, which indicates the enzymes responsible for this process may have high capacity. Accumulation of ala-AZT-MP at a given concentration in BT20 cells far exceeded that found in other cell lines. Peak intracellular concentration of the metabolite in BT20 cells was reached earliest among these cell lines and latest in Nalm6 cells. Together, these data suggest that conversion of Compound 003 to its metabolite occurs most rapidly in BT20 cells, while Nalm6 cells show the slowest rate of metabolism.

To study the reasons for the observed significant differences among these cell lines, uptake, efflux, half-life, enzyme activity, and further metabolism were compared. Efflux of ala-AZT-MP was only detectable in BT20 cells, which is consistent with drastic decrease in intracellular concentration of the metabolite in these cells after reaching peak concentration. There appears to be a threshold, over which the metabolite will be “spilled” outside the cells.

Uptake of Compound 003 in BT20 cells is about 2 fold higher than those in Nalm6 and CEM cells, and about 9 fold higher than T98 cells. Since nonradioactive compound was used in the study and rapid conversion of parent compound into the metabolite, an indirect method was used to measure the uptake of this compound into the cells, which mainly reflects the association of Compound 003 with these cells. A nine-fold difference in uptake between BT20 and T98 cells was consistent with the difference,in the accumulation of ala-AZT-MP with either intact cells or cell homogenates of,these two lines.

These results suggest that the uptake of the parent compound may be driven by intracellular metabolism. Since esterase is implicated to be involved in the first activation step of nucleoside phosphoramidates in the cell esterase activities were measured using p-nitrophenolacetate (pNPA) as the substrate. In agreement with higher intracellular Compound 003 metabolism in BT20 cells, these cells also had higher pNPA hydrolytical activity than T98 cells. This finding points to the importance of the first conversion step as the rate-limiting step of Compound -003 metabolism, and supports the role of esterases in the conversion of Compound 003 to ala-AZTMP. Preliminary results also showed that Compound 003 metabolism is inhibited by paraoxon, an inhibitor of esterases (McCracken, N. et al., (1993) supra).

While half-lives of ala-AZT-MP were similar between BT20 and T98 cells, ala-AZT-MP is preferentially converted to AZT-MP in BT20 cells, and to AZT in T98 cells. Although ala-AZT-MP to AZT conversion was proposed by others as a major pathway and to AZT-MP as a minor pathway of metabolism (McIntee, E. J., et al., (1997) J Med Chem. 40:3323; Balzarini, (1996) J., et al., Proc Natl Acad Sci U S A. 93:7295), we show here that this is not true for all cell types.

In addition, little AZT-MP was detected in T98 cells at either 3 hour or 24 h, showing slow conversion of AZT to AZT-MP. Neither AZT nor AZT-MP was detectable in BT20 cells at 24 hr, which may be due to low level of ala-AZT-MP at this time resulted from rapid metabolism and substrate depletion in the media after 24 hours (FIG. 12). The level of ala-AZT-MP formation was 2-fold higher in CEM than that in Nalm6 either in intact cells or cell homogenates. The uptake of the parent compound was similar in Nalm6 and CEM cells, so was pNPA hydrolytic activity in these two cells. However, esterases may have different substrate specificity towards pNPA and Compound 003, as suggested by reduced metabolic differences using pNPA as the substrate versus Compound 003 (3-fold vs. 9-fold) in BT20 cells and T98 cells. Therefore, subtle differences of Compound 003 metabolism in CEM and Nalm6 cells may not be reflected by pNPA hydrolysis.

On the other hand, the accumulation of ala-AZT-MP in these two cell lines may also be related to down-stream pathways for further metabolism. Nalm6 appears to have faster rate of conversion from AZT to AZT-MP, resulting in lower intracellular level of ala-AZT-MP. Take together, fast intracellular metabolism was observed in BT20 cells, which was contributed mainly by higher esterase activity in these cells, which was further support by esterase inhibition results.

It has been proposed that phosphoramidate derivatives of nucleosides are metabolized by carboxylesterases (McIntee, E. J., et al., (1997) supra; Valette, G., et al., (1996) J Med Chem. 39:1981). Many drugs require activation by carboxylesterases, including recently reported irinotecan (CPT-I 1) (Guichard, S. M., et al., (1998) Clin Cancer Res. 4:3089; Wang, L., et al., (1996) J Med Chem. 39:826). In this study, various esterase inhibitors were utilized to evaluate the role of-esterases in the metabolism of the AZT phosphoramidate DDE-3 to ala-AZT-MP. Our results show that Compound 003 is metabolized by carboxylesterases to form ala-AZT-MP, and these esterases were sensitive to paraoxon and other carboxylesterase inhibitors, but not to cholinesterase inhibitor, physostigmine, in Nalm6 and BT20 cells.

Although the metabolism was susceptible to paraoxon inhibition in both Nalm6 and BT20 cells, there are qualitative and quantitative differences in the inhibition profiles of these two cell lines. In BT20 cells, the potency of these esterase inhibitors were as follows: paraoxon>BNPP>PMSF>physostigmine. However, there was no such relationship observed with any of the compounds in Nalm6 cells, in which it seems as if only paraoxon was effective in inhibiting the metabolism. Quantitatively, the extent of inhibition was generally greater in BT20 cells than that in Nalm6 cells. These data suggest that there may be different carboxylesterases mediating the metabolism of these compounds in these two cell lines.

These experiments established the structures of the major phase I metabolites and pharmacokinetics of Compound 003 and its metabolites in mice. The structures of Compound 003-M1 and Compound 003-M2 were identified with ¹H-NMR, UV and LC-MS. Based on the molecular weight determination via MS and the corresponding fragmentation properties observed, as well as the NMR and UV analysis, the two major metabolites was identified as 3′-azido-3′-deoxythyrmidine 5′-alaninyl phosphate (ala-AZT-MP) and AZT.

Orally-administered Compound 003 yielded ala-AZT-MP and AZT as the two major metabolites. No Compound 003 was detectable in the blood after oral administration. This lack of Compound. 003 in the plasma may be attributed to several factors. First, Compound 003 was unstable in both gastric fluid and intestinal fluid, in addition to its instability in blood. Even though small amounts of residual intact Compound 003 may be absorbed in the stomach, it is likely-to be quickly hydrolyzed in blood. Compound 003 decomposes readily in intestinal fluid to form ala-AZT-MP. This metabolite is likely to be absorbed in the intestine and then further metabolized to yield AZT in the blood.

AZT has-been demonstrated to be very quickly eliminated (t_{1/2} ˜21.2 minutes) (Wang et al. (1996) supra. However, the elimination half-life of AZT following oral administration of Compound 003 was increased to 35.0 minutes following intravenous injection and to 148.7 minutes following oral administration of Compound 003. The exact mechanism for the observed longer elimination half-life of AZT after administration of the parent Compound 003 is not clear. Such a phenomenon has been was observed with metabolites of other compounds (See, for example, Chen, et al. (2001) Drug Metab Dispos. 29, 1035; Pang, et al. (1980) Drug Metab Dispos. 8, 39; Pang, K. S., (1981) J Pharmacokinet Biopharm. 9, 477; Houston, et al. (1984) Br J Clin Pharmacol. 17. 385; Lin, et al. (1984) J Pharm Sci. 73, 285.) The metabolism pathways for Compound 003 are similar to that of stampidine, an aryl phosphate derivative of d4T, which is being developed as an anti-HIV agent (See, for example, Uckun, et al. (2003). Antimicrob Agents Chemother. 47, 1233; Uckun, et al. (2003) Arzneimittelforschung. 53, 357; Uckun, et al. (2002) Antimicrob Agents Chemother. 46, 3428; Uckun, et al. (2002) Antimicrob Agents Chemother. 46, 3613; Uckun, et al. (2002) Antivir Chem Chemother. 13, 197.)

### REFERENCES

All references cited herein, including those listed below are hereby incorporated by reference into the specification.
[1] Barnes, D. M., Millis, R. R., Gillett, C. E., et al., The interaction of oestrogen receptor status and pathological features with adjuvant treatment in relation to survival in patients with operable breast cancer: a retrospective study of 2660 patients. Endocr Relat Cancer. 11, 85 (2004)
[2] Gee, J. M., Robertson, J. F., Ellis, I. O., et al., Phosphorylation of ERK1/2 mitogen-activated protein kinase is associated with poor response to anti-hormonal therapy and decreased patient survival in clinical breast cancer. Int J Cancer. 95, 247 (2001)
[3] Lindley, C., Developments in breast cancer therapy. J Am Pharm Assoc (Wash). 42, S30 (2002)
[4] Spigel, D. R., Burstein, H. J., Trastuzumab regimens for HER2-overexpressing metastatic breast cancer. Clin Breast Cancer. 4, 329 (2003)
[5] Tripathy, D., Slamon, D. J., Cobleigh, M., et al., Safety of treatment of metastatic breast cancer with trastuzumab beyond disease progression. J Clin Oncol. 22, 1063 (2004)
[6] Bonadonna, G., Zambetti, M., Bumma, C., et al., Multimodal treatment with primary single-agent epirubicin in operable breast cancer: 5-year experience of the Michelangelo Cooperative Group. Ann Oncol. 13, 1049 (2002)
[7] Bonadonna, G., Valagussa, P., Moliterni, A., et al., Adjuvant cyclophosphamide, methotrexate, and fluorouracil in node-positive breast cancer: the results of 20 years of follow-up. N Engl J Med. 332, 901 (1995)
[8] Ingle, J. N., Foley, J. F., Mailliard, J. A., et al., Randomized trial of cyclophosphamide, methotrexate, and 5-fluorouracil with or without estrogenic recruitment in women with metastatic breast cancer. Cancer. 73 2337(1994)
[9] Somlo, G., Doroshow, J. H., Forman, S. J., et al., High-dose doxorubicin, etoposide, and cyclophosphamide with stem cell reinfusion in patients with metastatic-or high-risk primary breast cancer. City of Hope Bone Marrow Oncology Team. Cancer. 73, 1678 (1994)
[10] Somlo, G., Doroshow, J. H., Synold, T., et al., High-dose paclitaxel in combination with doxorubicin, cyclophosphamide and peripheral blood progenitor cell rescue in patients with high-risk primary and responding metastatic breast carcinoma: toxicity profile, relationship to paclitaxel pharmacokinetics and short-term outcome. Br J Cancer. 84, 1591 (2001)
[11] Scheer, M., Prange, W., Petmecky, K., et al., [Evaluation of her-2/neu amplification/overexpression in OSCC with fluorescence in situ hybridization (FISH) and immunohistochemistry]. Mund Kiefer Gesichtschir. 7, 138 (2003)
[12] Earp, H. S., 3rd, Calvo, B. F., Sartor, C. I., The EGF receptor family—multiple roles in proliferation, differentiation, and neoplasia with an emphasis on HER4. Trans Am Clin Climatol Assoc. 114, 315 (2003)
[13] Azria, D., Larbouret, C., Robert, B., et al., [Radiotherapy and inhibitors of epidermal growth factor receptor: preclinical findings and preliminary clinical trials]. Bull Cancer. 90 Spec No, S202 (2003)
[14] Albanell, J., Codony, J., Rovira, A., et al., Mechanism of action of anti-HER2 monoclonal antibodies: scientific update on trastuzumab and 2C4. Adv Exp Med Biol. 532, 253 (2003)
15] Navolanic, P. M., Steelman, L. S., McCubrcy, J. A., EGFR family signaling and its association with breast cancer development and resistance to chemotherapy (Review) Int J Oncol. 22, 237 (2003)
[16] Zhu, X. F., Liu, Z. C., Xie, B. F., et al., [SUCI02 inhibits HER-2/neu receptor tyrosine kinase phosphorylation and growth of HER-2/neu-overexpressing breast cancer cells]. Ai Zheng. 22, 790 (2003)
[17] Witton, C. J., Reeves, J. R., Going, J. J., et al., Expression of the HER1-4 family of receptor tyrosine kinases in breast cancer. J Pathol. 200, 290 (2003)
[18] Major, M. A., Clinical trials update: Medical management of advanced breast cancer. Cancer Nurs. 26, 10S (2003)
[19] Yoh, K., Ishii, G., Yokose, T., et al., Breast cancer resistance protein impacts clinical outcome in platinum-based chemotherapy for advanced non-small cell lung cancer. Clin Cancer Res. 10, 1691 (2004)
[20] Yang, Z., Bagheri-Yarmand, R., Wang, R. A., et al., The epidermal growth factor receptor tyrosine kinase inhibitor ZD1 839 (Iressa) suppresses c-Src and Pak1 pathways and invasiveness of human cancer cells. Clin Cancer Res. 10, 658 (2004)
[21] Pedersen, M. W., Tkach, V., Pedersen, N., et al., Expression of a naturally occurring constitutively active variant of the epidermal growth factor receptor in mouse fibroblasts increases motility. Int J Cancer. 108, 643 (2004)
[22] Wagner, C. R., Ballato, G., Akanni, A. O., et al., Potent growth inhibitory activity of zidovudine on cultured human breast cancer cells and rat mammary tumors. Cancer Res. 57, 2341 (1997)
[23] Iyer, V. V., Griesgraber, G. W., Radmer, M. R., et al., Synthesis, in vitro anti-breast cancer activity, and intracellular decomposition of amino acid methyl ester and alkyl amide phosphoramidate monoesters of 3′-azido-3′-deoxythymidine (AZT). J Med Chem. 43,2266(2000)
[24] Melana, S. M., Holland, J. F., Pogo, B. G., Inhibition of cell growth and telomerase activity of breast cancer cells in vitro by 3′-azido-3′-deoxythymidine. Clin Cancer Res. 4,693(1998)
[25] Brown, Sigurdson, E., Rogatko, A., et al., Telomerase inhibition using azidothymidine in the HT-29 colon cancer cell line. Ann Surg Oncol. 10, 910 (2003)
[26] Sharma, M., Jain, R., Ionescu, E., et al., Quantitative analysis of 3′-azido-3-deoxythymidine incorporation into DNA in human colon tumor cells. Bioconjug Chem. 6, 536(1995)
[27] Darnowski, J. W., Goulette, F. A., 3′-Azido-3′-deoxythymidine cytotoxicity and metabolism in the human colon tumor cell line HCT-8. Biochem Pharmacol. 48, 1797 (1994)
[28] Brunetti, I., Falcone, A., Calabresi, P., et al., 5-Fluorouracil enhances azidothymidine cytotoxicity: in vitro, in vivo, and biochemical studies. Cancer Res. 50, 4026(1990)
[29] Weber, G., Nagai, M., Prajda, N., et al., AZT: a biochemical response modifier of methotrexate and 5-fluorouracil cytotoxicity in human ovarian and pancreatic carcinoma cells. Cancer Commun. 3, 127 (1991)
[30] Yasuda, C., Kato, M., Kuroda, D., et al., Experimental studies on potentiation of the antitumor activity of 5-fluorouracil with 3′-azido-3′-deoxythymidine for the gastric cancer cell line MKN28 in vivo. Jpn J Cancer Res. 88, 97 (1997)
[31] Weber, G., Ichikawa, S., Nagai, M., et al., Azidothymidine inhibition of thymidine kinase and synergistic cytotoxicity with methotrexate and 5-fluorouracil in rat hepatoma and human colon cancer cells. Cancer Commun. 2, 129 (1990)
[32] Johnston, J. S., Johnson, A., Gan, Y., et al., Synergy between 3′-azido-3′-deoxythymidine and paclitaxel in human pharynx FaDu cells. Pharm Res. 20, 957 (2003)
[33] Hermine, O., Allard, I., Levy, V., et al., A prospective phase II clinical trial with the use of zidovudine and interferon-alpha in the acute and lymphoma forms of adult T-cell leukemia/lymphoma. Hematol J. 3, 276 (2002)
[34] Tosi, P., Visani, G., Ottaviani, E., et al., Synergistic cytotoxicity of AZT plus alpha and gamma interferon in chronic myeloid leukemia cell line K562. Eur J Haematol. 51, 209 (1993)
[35] Gomez, D. E., Tejera, A. M., Olivero, O. A., Irreversible telomere shortening by, 3′-azido-2′,3′-dideoxythymidine (AZT) treatment. Biochem Biophys Res Commun. 246, 107 (1998)
[36] Morgan, R. J., Jr., Newman, E. M., Sowers, L., et al., Phase I study of cisdiamminedichloroplatinum in combination with azidothymidine in the treatment of patients with advanced malignancies. Cancer Chemother Pharmacol. 51, 459 (2003)
[37] McGuigan, C., Pathirana, R. N., Balzarini, J., et al., Intracellular delivery of bioactive AZT nucleotides by aryl phosphate derivatives of AZT. J Med Chem. 36, 1048 (1993)
[38] McGuigan, C., Cahard, D., Sheeka, H. M., et al., Aryl phosphoramidate derivatives of d4T have improved anti-HIV efficacy in tissue culture and may act by the generation of a novel intracellular metabolite. J Med Chem. 39, 1748 (1996)
[39] Egron, D., Lefebvre, I., Perigaud, C., et al., Anti-HIV pronucleotides: decomposition pathways and correlation with biological activities. Bioorg Med Chem Lett. 8, 1045 (1998)
[40] Moudjou, M., Bornens, B., Cell Biology: A Laboratory Handbook, p. 595. Academic Press, Inc, New York (1994)
[41] Cell Biology: A Laboratory Handbook, (1994)
[42] Navara, C. S., Benyumov, A., Vassilev, A., et al., Vanadocenes as potent anti-proliferative agents disrupting mitotic spindle formation in cancer cells. Anticancer Drugs. 12, 369 (2001)
[43] Uckun, F. M., Tuel-Ahlgren, L., Waddick, K. G., et al., Physical and functional interactions between Lyn and p34cdc2 kinases in irradiated human B-cell precursors. J Biol Chem. 271, 6389 (1996)
[44] Malaviya, R., Navara, C., Uckun, F. M., Augmentation of mast cell bactericidal activity by the anti-leukemic drug, 4-(3′bromo-4′-hydroxylphenyl)-amino-6%,7-dimethoxyquinazoline. Leuk Lymphoma. 43, 1329 (2002)
[45] Ghosh, S., Zheng, Y., Jun, X., et al., Alpha-cyano-beta-hydroxy-beta-methyl-N-[4-(trifluoromethoxy)phenyl] propenamide: an inhibitor of the epidermal growth factor receptor tyrosine kinase with potent cytotoxic activity against breast cancer cells. Clin Cancer Res. 4, 2657 (1998)
[46] Uckun, F. M., Chen, C. L., Lisowski, E., et al., Toxicity and pharmacokinetics of stampidine in mice and rats. Arzneimittelforschung. 53, 357 (2003)
[47] Uckun, F. M., Zheng, Y., Cetkovic-Cvrlje, M., et al., In vivo pharmacokinetic features, toxicity profile, and chemosensitizing activity of alpha-cyano-beta-hydroxy-beta-methyl-N-(2,5-dibromophenyl)propenamide (LFM-A13), a novel antileukemic agent targeting Bruton's tyrosine kinase. Clin Cancer Res. 8, 1224 (2002)
[48] Uckun, F. M., Qazi, S., Pendergrass, S., et al., In vivo toxicity, pharmacokinetics, and anti-human immunodeficiency virus activity of stavudine-5′-(p-bromophenyl methoxyalaninyl phosphate) (stampidine) in mice. Antimicrob Agents Chemother. 46, 3428 (2002)
[49] Treco, D. A., 5th, p. 2.1.1. John Wiley and Sons, New York (1995)
[50] Werneburg, N., Guicciardi, M. E., Yin, X. M., et al., Tumor Necrosis Factor-{alpha}-Mediated Lysosomal Permeabilization Is FAN and Caspase 8/Bid-Dependent. Am J Physiol Gastrointest Liver Physiol. (2004)
[51] Cailleret, M., Amadou, A., Andrieu-Abadie, N., et al., N-acetylcysteine prevents the deleterious effect of tumor necrosis factor-(alpha) on calcium transients and contraction in adult rat cardiomyocytes. Circulation. 109, 406 (2004)
[52] Asefa, B., Klarmann, K. D., Copeland, N. G., et al., The interferon-inducible p200 family of proteins: a perspective on their roles in cell cycle regulation and differentiation. Blood Cells Mol Dis. 32, 155 (2004)
[53] Terrinoni, A., Ranalli, M., Cadot, B., et al., p73-alpha is capable of inducing scotin and ER stress. Oncogene. 23, 3721 (2004)
[54] Ghribi, O., Herman, M. M., Pramoonjago, P., et al., MPP+ induces the endoplasmic reticulum stress response in rabbit brain involving activation of the ATF-6 and NF-kappaB signaling pathways. J Neuropathol Exp Neurol. 62, 1144 (2003).
[55] Wagner, C. R., Ballato, G., Akanni, A. O., et al., Potent growth inhibitory activity of zidovudine on cultured human breast cancer cells and rat mammary tumors. Cancer Res. 57, 2341 (1997)
[56] Iyer, V. V., Griesgraber, G. W., Radmer, M. R., et al., Synthesis, in vitro anti-breast cancer activity, and intracellular decomposition of amino acid methyl ester and alkyl amide phosphoramidate monoesters of 3′-azido-3′-deoxythymidine (AZT). J Med Chem. 43, 2266 (2000)
[57] Melana, S. M., Holland, J. F., Pogo, B. G., Inhibition of cell growth and telomerase activity of breast cancer cells in vitro by 3′-azido-3′-deoxythymidine. Clin Cancer Res. 4,693(1998)
[58] Brown, T., Sigurdson, E., Rogatko, A., et al., Telomerase inhibition using azidothymidine in the HT-29 colon cancer cell line. Ann Surg Oncol. 10, 910 (2003)
[59] Sharma, M., Jain, R., Ionescu, E., et al., Quantitative analysis of 3′-azido-3′-deoxythymidine incorporation into DNA in human colon tumor cells. Bioconjug Chem. 6,536(1995)
[60] Darnowski, J. W., Goulette, F. A., 3′-Azido-3′-deoxythymidine cytotoxicity and metabolism in the human colon tumor cell line HCT-8. Biochem Pharmacol. 48, 1797 (1994)
[61] Brunetti, I., Falcone, A., Calabresi, P., et al., 5-Fluorouracil enhances azidothymidine cytotoxicity: in vitro, in vivo, and biochemical studies. Cancer Res. 50), 4026 (1990)
[62] Weber, G., Nagai, M., Prajda, N., et al., AZT: a biochemical response modifier of methotrexate and 5-fluorouracil cytotoxicity in human ovarian and pancreatic carcinoma cells. Cancer Commun. 3, 127 (1991)
[63] Yasuda, C., Kato, M., Kuroda, D., et al., Experimental studies on potentiation of the antitumor activity of 5-fluorouracil with 3′-azido-3′-deoxythymidine for the gastric cancer cell line MKN28 in vivo. Jpn J Cancer Res. 88, 97 (1997)
[64] Weber, G., Ichikawa, S., Nagai, M., et al., Azidothymidine inhibition of thymidine kinase and synergistic cytotoxicity with methotrexate and 5-fluorouracil in rat hepatoma and human colon cancer cells. Cancer Commun. 2, 129 (1990)
[65] Johnston, J. S., Johnson, A., Gan, Y., et al., Synergy between 3′-azido-3′-deoxythymidine and paclitaxel in human pharynx FaDu cells. Pharm Res. 20, 957 (2003)
[66] Hermine, O., Allard, I., Levy, V., et al., A prospective phase II clinical trial with the use of zidovudine and interferon-alpha in the acute and lymphoma forms of adult T-cell leukemia/lymphoma. Hematol J. 3, 276 (2002)
[67] Tosi, P., Visani, G., Ottaviani, E., et al., Synergistic cytotoxicity of AZT plus alpha and gamma interferon in chronic myeloid leukemia cell line K562. Eur J Haematol. 51, 209 (1993)
[68]Gomez, D. E., Tejera, A. M., Olivero, 0. A., Irreversible telomere shortening by 3′-azido-2′,3′-dideoxythymidine (AZT) treatment. Biochem Biophys Res Commun. 246, 107 (1998)
[69] Morgan, R. J., Jr., Newman, E. M., Sowers, L., et al., Phase I study of cisdiamminedichloroplatinum in combination with azidothymidine in the treatment of patients with advanced malignancies. Cancer Chemother Pharmacol. 51, 459 (2003)
[70]
[71] Egron, D., Lefebvre, I., Perigaud, C., et al., Anti-HIV pronucleotides:

decomposition pathways and correlation with biological activities. Bioorg Med Chem Lett. 8, 1045 (1998)
[72] McGuigan, C., Pathirana, R. N., Balzarini, J., et al., Intracellular delivery of bioactive AZT nucleotides by aryl phosphate derivatives of AZT. J Med Chem. 36, 1048 (1993)
[73] McGuigan, C., Cahard, D., Sheeka, H. M., et al., Aryl phosphoramidate derivatives of d4T have improved anti-HIV efficacy in tissue culture and may act by the generation of a novel intracellular metabolite. J Med Chem. 39, 1748 (1996)
[74] McGuigan, C., Cahard, D., Sheeka, H. M., et al., Phosphoramidate derivatives of d4T with improved anti-HIV efficacy retain full activity in thyrmidine kinase-deficient cells: Bioorg Med Chem Lett. 6, 1183 (1996)
[75] Chen, C. L., Chen, H., Zhu, D. M., et al., Quantitative high-performance liquid chromatography-based detection method for calphostin C, a naturally occurring perylenequinone with potent antileukemic activity. J Chromatogr B Biomed Sci Appl. 724. 157 (1999)
[76] Chen, C. L., Malaviya, R., Chen, H., et al., Quantitative high-performance liquid chromatographic method for pharmacokinetic studies of the potent mast cell inhibitor 4-(4′-hydroxyphenyl)amino-6,7-dimethoxyquinazoline (WHI-P 131). J Chromatogr B Biomed Sci Appl. 727, 205 (1999)
[77] Chen, C. L., Malaviya, R., Navara, C., et al., Pharmacokinetics and biologic activity of the novel mast cell inhibitor, 4-(3-hydroxyphenyl)-amino-6,7-dimethoxyquinazoline in mice. Pharm Res. 16, 117 (1999)
[78] Chen, C. L., Tai, H. L., Zhu, D. M., et al., Pharmacokinetic features and metabolism of calphostin C, a naturally-occurring perylenequinone with antileukemic activity. Pharm Res. 16, 1003 (1999)
[79] Uckun, F. M., Ek, O., Liu, X. P., et al., In vivo toxicity and pharmacokinetic features of the janus kinase 3 inhibitor WHI-P131 [4-(4′hydroxyphenyl)-amino-6,7-dimethoxyquinazoline. Clin Cancer Res. 5, 2954 (1999)
[80] Morgan, E. W., Yan, B., Greenway, D., et al., Purification and characterization of two rat liver microsomal carboxylesterases (hydrolase A and B). Arch Biochem Biophys. 315, 495 (1994)
[81] McCracken, N. W., Blain, P. G., Williams, F. M., Human xenobiotic metabolizing esterases in liver and blood. Biochem Pharmacol. 46, 1125 (1993)
[82] Gaustad, R., Johnsen, H., Fonnum, F., Carboxylesterases in guinea pig. A comparison of the different isoenzymes with regard to inhibition by organophosphorus compounds in vivo and in vitro. Biochem Pharmacol. 42, 1335 (1991)
[83] Kaliste-Korhonen, E., Tuovinen; K., Hanninen, O., Interspecies differences in enzymes reacting with organophosphates and their inhibition by paraoxon in vitro. Hum Exp Toxicol. 15, 972 (1996)
[84] Ehrich, M., Correll, L., Inhibition of carboxylesterases in SH-SY5Y human and NB41A3 mouse neuroblastoma cells by organophosphorus esters. J Toxicol Environ Health A. 53, 385 (1998)
[85] Williams, F. M., Clinical significance of esterases in man. Clin Pharmacokinet. 10, 392(1985)
[86] McIntee, E. J., Remmel, R. P., Schinazi, R. F., et al., Probing the mechanism of action and decomposition of amino acid phosphomonoester amidates of antiviral nucleoside prodrugs. J Med Chem. 40, 3323 (1997)
[87] Balzarini, J., Karlsson, A., Aquaro, S., et al., Mechanism of anti-HIV action of masked alaninyl d4T-MP derivatives. Proc Natl Acad Sci U S A. 93, 7295 (1996)
[88] Valette, G., Pompon, A., Girardet, J. L., et al., Decomposition pathways and in vitro HIV inhibitory effects of isoddA pronucleotides: toward a rational approach for intracellular delivery of nucleoside 5′-monophosphates. J Med Chem. 39, 1981 (1996)
[89] Guichard, S. M., Morton, C. L., Krull, E. J., et al., Conversion of the CPT-11 metabolite APC to SN-38 by rabbit liver carboxylesterase. Clin Cancer Res. 4, 3089 (1998)
[90] Wang, L., Morin, K. W., Kumar, R., et al., In vivo biodistribution, pharmacokinetic parameters, and brain uptake of 5-halo-y-methoxy(or ethoxy)-5,6-dihydro-3′-azido-3′-deoxythymidine diastereomers as potential prodrugs of 3′-azido-3′-deoxythymidine. J Med Chem. 39, 826.(1996)
[91] Chen, C. L., Venkatachalam, T. K., Zhu, Z. H., et al., In vivo pharmacokinetics and metabolism of anti-human immunodeficiency virus agent D4T-5′-[p-bromophenyl methoxyalaninyl phosphate] (SAMPIDINE) in mice. Drug Metab Dispos. 29, 1035 (2001)
[92] Pang, K. S., Gillette, J. R., Metabolite pharmacokinetics: methods for simultaneous estimates of elimination rate constants of a drug and its metabolite. A commentary. Drug Metab Dispos. 8, 39 (1980)
[93] Pang, K. S., Metabolite pharmacokinetics: the area under the curve of metabolite and the fractional rate of metabolism of a drug after different routes of administration for renally and hepatically cleared drugs and metabolites. J Pharmacokinet Biopharm. 9, 477 (1981)
[94] Houston, J. B., Taylor, G., Drug metabolite concentration-time profiles: influence of route of drug administration. Br J Clin Pharmacol. 17, 385 (1984)
[95] Lin, H., Levy, R. H., Lane, E. A., et al., Variability in the determination of fraction metabolized in a triangular metabolic problem and its resolution with stable isotope methodology. J Pharm Sci. 73, 285 (1984)
[96] Uckun, F. M., Chen, C. L., Samuel, P., et al., In vivo antiretroviral activity of stampidine in chronically feline immunodeficiency virus-infected cats. Antimicrob Agents Chemother. 47, 1233 (2003)
[97] Uckun, F. M., Chen, C. L., Lisowski, E., et al., Toxicity and pharmacokinetics of stampidine in mice and rats. Arzneimittelforschung. 53, 357 (2003)
[98] Uckun, F. M., Qazi, S., Pendergrass, S., et al., In vivo toxicity, pharmacokinetics, and anti-human immunodeficiency virus activity of stavudine-5′-(p-bromophenyl methoxyalaninyl phosphate) (stampidine) in mice. Antimicrob Agents Chemother. 46, 3428 (2002)
[99] Uckun, F. M., Pendergrass, S., Venkatachalam, T. K., et al., Stampidine is a potent inhibitor of Zidovudine- and nucleoside analog reverse transcriptase inhibitor-resistant primary clinical human immunodeficiency virus type 1 isolates with thymidine analog mutations. Antimicrob Agents Chemother. 46, 3613 (2002)
[100] Uckun, F. M., Samuel, P., Qazi, S., et al., Effects of aryl substituents on the anti-HIV activity of the arylphosphoramidate derivatives of stavudine. Antivir Chem Chemother. 13, 197 (2002)
[101] Aryl phosphate derivatives of D4T having anti-HIV activity, U.S. Pat. No. 6,670,336. Issued. Dec. 30, 2003. “Aryl phosphate derivatives of d4T having anti-HIV activity.” Inventors: Uckun; Fatih M.; Vig; Rakesh.

1. A pharmaceutical composition comprising a compound of the formula: 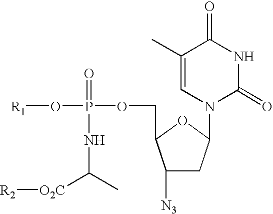
where R₁ is H or 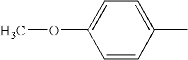
R₂ is H or Me;
or a pharmaceutically acceptable salt thereof; and
one or more carboxylesterase inhibitors.

2. The pharmaceutical composition of claim 1, wherein at least one of the carboxylesterase inhibitors is paraoxon.

3. The pharmaceutical composition comprising a compound of claim 2 in combination with one or more pharmaceutically acceptable carriers, diluents, or adjuvants.

4. The pharmaceutical composition of claim 1, wherein the compound is

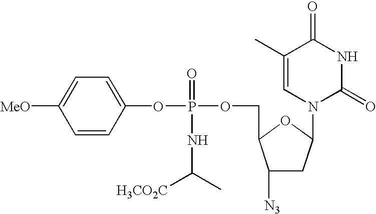

5. A pharmaceutical composition comprising a compound of the formula: 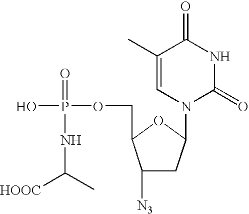
or a pharmaceutically acceptable salt thereof; and
a pharmaceutically acceptable carrier, diluent, or adjuvant.

6. A method inhibiting cellular proliferation associated with proliferative cell disorders in a subject comprising:
administering to the subject a compound of the formula: 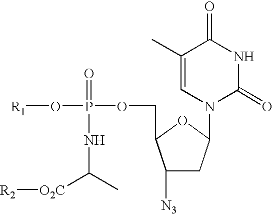
where R₁ is H or 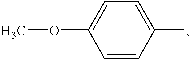
and
R₂ is H or Me;
or a pharmaceutically acceptable salt thereof.

7. The method of claim 6, wherein the compound is

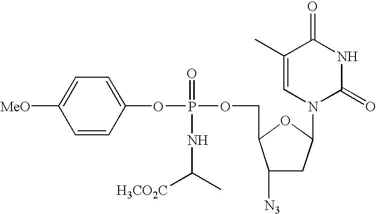

8. The method of claim 7, additionally comprising administering at least one carboxylesterase inhibitor.

9. The method of claim 8, wherein the carboxylesterase inhibitor is paraoxon.

10. The method of claim 8, wherein the carboxylesterase inhibitor is administered concurrently with the compound.

11. A method for arresting the cell cycle in actively dividing cells comprising:
administering to the actively dividing cells a compound of the formula: 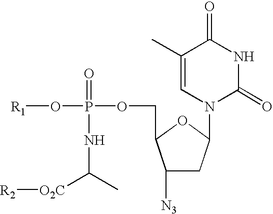
where R₁ is H or 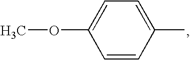
R₂ H or Me;
o r a pharmaceutically acceptable salt thereof.

12. The method of claim 11, wherein the compound is

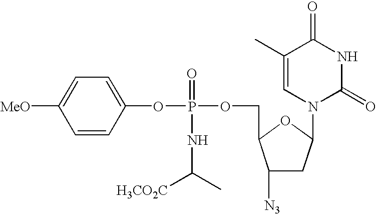

13. The method of claim 12, additionally comprising administering at least one carboxylesterase inhibitors.

14. The method of claim 13, wherein the carboxylesterase inhibitor is paraoxon.

15. The method of claim 13, wherein the carboxylesterase inhibitor is administered concurrently with the compound.

16. The method of claim 11, wherein the actively dividing cells are cancer cells.

17. The method of claim 11, wherein the actively dividing cells are human breast cancer cells.

18. The method of claim 11, further comprising inhibiting mitotic spindle formation in the actively dividing cells.


SEQUENCE LISTING (4 sequences)

SEQ ID NO: 1 - DNA, 30 bp, Artificial Sequence
  Primer
ccgggcagccaggtccctgtgtacaagccg

SEQ ID NO: 2 - DNA, 25 bp, Artificial Sequence
  Primer
ggaagtacccggatgagggcatatg

SEQ ID NO: 3 - DNA, 21 bp, Artificial Sequence
  Primer
gatgtgctgcaggctaaagtt

SEQ ID NO: 4 - DNA, 21 bp, Artificial Sequence
  Primer
agaaacggaatgttgtggagt